(19) United States
(12) Patent Application Publication
(10) Pub. No.: US 2026/0085039 A1
(43) Pub. Date: Mar. 26, 2026

(54) Psilocin crystalline forms

(71) Applicant: TRYP THERAPEUTICS INC., San Diego, CA (US)

(72) Inventors: Jim GILLIGAN, San Diego, CA (US); Peter GUZZO, San Diego, CA (US)

(21) Appl. No.: 19/110,733

(22) PCT Filed: Sep. 12, 2023
(86) PCT No.: PCT/IB2023/059011

§ 371 Date: Mar. 11, 2025

Related U.S. Application Data
(60) Provisional application No. 63/375,305, filed on Sep. 12, 2022.

(51) Int. Cl. C07D 209/16; A61K 31/4045; C07C 53/124; C07C 55/10; C07C 57/15; C07C 59/255; C07C 59/265; C07C 63/08; C07C 309/29

(52) U.S. Cl. CPC C07D 209/16; A61K 31/4045; C07C 53/124; C07C 55/10; C07C 57/15; C07C 59/255; C07C 59/265; C07C 63/08; C07C 309/29; C07B 2200/13

(57) ABSTRACT

The present invention relates to crystalline forms of psilocin (4-hydroxy-N,N-dimethyltryptamine) salts or cocrystals, as well as compositions, methods of preparation and methods of their use. The present invention also relates to said crystalline forms having improved physical properties such as aqueous solubility and stability, wherein the crystalline forms and compositions thereof are suitable for oral, subcutaneous, intravenous, or intramuscular administration.

### CROSS REFERENCE TO RELATED APPLICATION

This application claims priority to U.S. provisional patent application No. 63/375,305, filed 12 Sep. 2022, which is incorporated by reference in its entirety.

### TECHNICAL FIELD

The present invention relates to psilocin crystalline forms. In particular, the present invention relates to psilocin crystalline forms having improved physical properties such as aqueous solubility and stability.

### BACKGROUND OF THE INVENTION

Psilocin (4-hydroxy-N,N-dimethyltryptamine) is a psychoactive compound which is naturally-occurring, and may be isolated from psilocybin mushrooms. In vivo psilocybin is rapidly dephosphorylated to psilocin which is the psychoactive compound. Research into the therapeutic benefits of psilocybin and its active metabolite psilocin has led to the use of these psychoactives for the treatment of a variety of conditions including drug dependence, anxiety, depression, PTSD and eating disorders and chronic pain.

Both psilocin and psilocybin have limited stability in aqueous solutions and such solutions rapidly degrade on exposure to light. Moreover, the active agent psilocin has a relatively low solubility in aqueous media, which limits its ability to be used in, for example a dosage form suitable for intravenous or subcutaneous injection.

Due to the potentially variable effect of psilocin on each individual and the variability in absorption after oral dosing absorption, it is difficult to provide accurate doses with predictable effects. Moreover, the onset of the therapeutic effects after oral dosing are typically slow, beginning from up to about 40 minutes after administration, with the peak effects often not observed until hours after administration. Intravenous formulations of psilocin are desirable as they have the potential to provide standardisation of interindividual variability in plasma concentrations and faster onset of action. IV administration can also control the duration of the psychedelic experience.

Moreover, the increasing prevalence of and research into “microdosing” (i.e., administering psilocin or psilocybin in quantities much lower than typical therapeutic or recreational doses) means there is a need to produce psilocin formulations which can reliably be used to accurately administer low doses, for example maintenance doses.

Crystalline or amorphous solid forms of a pharmaceutically active agents can exist as single-component and multiple-component solids. Single-component solids consist essentially of the agent in the absence of other substances. Single-component crystalline materials may exist as different polymorphs, which have different three-dimensional arrangements of the component. Importantly, different polymorphs may have differing properties such as stability, solubility, melting point, reactivity, and other processability variations.

Multiple-component solids comprising two or more ionic species are referred to as salts. A pharmaceutical active or its salt may also exist in forms such as hydrates, solvates or cocrystals. Multiple-component crystal forms may also exhibit polymorphism if the components exist in more than one three-dimensional crystalline arrangement, with each form exhibiting potentially different physical properties.

Cocrystals are crystalline molecular complexes of two or more compounds bound together in a crystal lattice by non-ionic interactions. Pharmaceutical cocrystals are cocrystals of an active agent and one or more compounds referred to as coformers. Typical coformers include non-toxic pharmaceutically acceptable substances, such as a food additives, preservatives, pharmaceutical excipients, or other active agents.

For at least the reasons above, there is a need to produce psilocin in the form of salts or cocrystals having improved aqueous solubility and/or stability.

### SUMMARY

According to a first aspect, the invention provides a crystalline form of a pharmaceutically acceptable salt of psilocin (4-hydroxy-N,N-dimethyltryptamine), or a cocrystal of psilocin (4-hydroxy-N,N-dimethyltryptamine) and a coformer. In one embodiment the pharmaceutically acceptable salt is an acid.

The acid or coformer may be selected from one or more of acetic acid, aconitic acid, ascorbic acid, benzenesulfonic acid, benzoic acid, butyric acid, citric acid, erythorbic acid, fumaric acid, gentisic acid, glutamic acid, glycolic acid, hydrochloric acid, maleic acid, phosphoric acid, pyroglutamic acid, sorbic acid, succinic acid, sulfuric acid, tartaric acid, arginine, lysine, methyl paraben, nicotinamide and ethyl acetate.

In one embodiment the acid is benzenesulfonic acid. The crystalline form may be Besylate Form A.

The besylate Form A may exhibits XRPD (X-ray power diffraction) peaks at about 15.44° 2θ±0.20, 18.33° 2θ±0.20, and 25.41° 2θ±0.20. or exhibit peaks at about 14.73° 2θ±0.20, 15.44° 2θ±0.20, 18.33° 2θ±0.20, 22.59° 2θ±0.20, and 25.41° 2θ±0.20; or exhibit peaks at about 11.72° 2θ±0.20, 12.47° 2θ±0.20, 13.49° 2θ±0.20, 14.73° 2θ±0.20, 15.44° 2θ±0.20, 18.33° 2θ±0.20, 20.62° 2θ±0.20, 20.99° 2θ±0.20, 21.77° 2θ±0.20, 22.25° 2θ±0.20, 22.59° 2θ±0.20, 23.22° 2θ±0.20, 23.71° 2θ±0.20, 24.10° 2θ±0.20, and 25.41° 2θ±0.20.

In one embodiment the Besylate Form A may exhibit XRPD peaks at about 7.69° 2θ±0.20, 10.26° 2θ±0.20, 10.96° 2θ±0.20, 11.72° 2θ±0.20, 12.47° 2θ±0.20, 12.84° 2θ±0.20, 13.49° 2θ±0.20, 14.73° 2θ±0.20, 15.44° 2θ±0.20, 16.18° 2θ±0.20, 18.33° 2θ±0.20, 19.04° 2θ±0.20, 19.68° 2θ±0.20, 20.62° 2θ±0.20, 20.99° 2θ±0.20, 21.77° 2θ±0.20, 22.25° 2θ±0.20, 22.59° 2θ±0.20, 23.22° 2θ±0.20, 23.71° 2θ±0.20, 24.10° 2θ±0.20, 25.15° 2θ±0.20, 25.41° 2θ±0.20, 25.65° 2θ±0.20, 26.29° 2θ±0.20, 26.76° 2θ±0.20, 27.72° 2θ±0.20, 27.99° 2θ±0.20, 28.67° 2θ±0.20, 28.93° 2θ±0.20, 29.63° 2θ±0.20, 30.43° 2θ±0.20, 30.76° 2θ±0.20, 31.15° 2θ±0.20, 31.77° 2θ±0.20, 32.13° 2θ±0.20, 32.94° 2θ±0.20, 33.65° 2θ±0.20, 34.94° 2θ±0.20, 35.69° 2θ±0.20, and 36.49° 2θ±0.20

In another embodiment the Besylate Form A is characterized by an X-ray powder diffraction spectrum substantially as depicted in FIG. 4.

In another embodiment the acid is butyric acid. The crystalline form may be Butyrate Form A.

The Butyrate Form A may exhibit XRPD (X-ray power diffraction) peaks at about 13.24° 2θ±0.20, 15.34° 2θ±0.20, and 15.88° 2θ±0.20; or may exhibit peaks at about 13.24° 2θ±0.20, 15.34° 2θ±0.20, 15.88° 2θ±0.20, 16.28° 2θ±0.20, 20.95° 2θ±0.20, and 27.79° 2θ±0.20; or may exhibit XRPD (X-ray power diffraction) peaks at about 9.33° 2θ±0.20, 9.96 26±0.20, 10.66° 2θ±0.20, 13.24° 2θ±0.20, 15.34° 2θ±0.20, 15.88° 2θ±0.20, 16.28° 2θ±0.20, 17.80° 2θ±0.20, 20.95° 2θ±0.20, 21.98° 2θ±0.20, 22.32° 2θ±0.20, 23.31° 2θ±0.20, 24.61° 2θ±0.20, and 27.79° 2θ±0.20.

In one embodiment the Butyrate Form A may exhibit XRPD peaks at about 9.33° 2θ±0.20, 9.96° 2θ±0.20, 10.66° 2θ±0.20, 12.96° 2θ±0.20, 13.24° 2θ±0.20, 14.36° 2θ±0.20, 15.34° 2θ±0.20, 15.88° 2θ±0.20, 16.28° 2θ±0.20, 17.80° 2θ±0.20, 18.36° 2θ±0.20, 18.75° 2θ±0.20, 18.97° 2θ±0.20, 19.63° 2θ±0.20, 20.01° 2θ±0.20, 20.35° 2θ±0.20, 20.95° 2θ±0.20, 21.44° 2θ±0.20, 21.98° 2θ±0.20, 22.32° 2θ±0.20, 22.80° 2θ±0.20, 23.31° 2θ±0.20, 23.77° 2θ±0.20, 24.41° 2θ±0.20, 24.61° 2θ±0.20, 25.46° 2θ±0.20, 25.60° 2θ±0.20, 26.22° 2θ±0.20, 26.67° 2θ±0.20, 27.79° 2θ±0.20, and 29.07° 2θ±0.20.

In another embodiment the Butyrate Form A may be characterized by an X-ray powder diffraction spectrum substantially as depicted in FIG. 7.

In another embodiment the acid is gentisic acid. The crystalline form is Gentisate Form A.

The Gentisate Form A may exhibits XRPD peaks at about 15.80° 2θ±0.20, 16.51° 2θ±0.20, and 23.98° 2θ±0.20, or may exhibits XRPD peaks at about 15.52° 2θ±0.20, 15.80° 2θ±0.20, 16.51° 2θ±0.20, 23.98° 2θ±0.20, and 24.74° 2θ±0.20; or may exhibit XRPD peaks at about 12.77° 2θ±0.20, 14.08° 2θ±0.20, 15.52° 2θ±0.20, 15.80° 2θ±0.20, 15.98±0.20, 16.51° 2θ±0.20, 17.30° 2θ±0.20, 18.58° 2θ±0.20, 20.95° 2θ±0.20, 21.64° 2θ±0.20, 23.38° 2θ±0.20, 23.98° 2θ±0.20, 24.74° 2θ±0.20, 25.19° 2θ±0.20, 27.81° 2θ±0.20, 28.41° 2θ±0.20, and 28.80° 2θ±0.20.

In one embodiment Gentisate Form A and exhibits XRPD peaks at about 7.74° 2θ±0.20, 9.01° 2θ±0.20, 11.01° 2θ±0.20, 12.29° 2θ±0.20, 12.77° 2θ±0.20, 13.15° 2θ±0.20, 13.80° 2θ±0.20, 14.08° 2θ±0.20, 15.52° 2θ±0.20, 15.80° 2θ±0.20, 15.98° 2θ±0.20, 16.11° 2θ±0.20, 16.51° 2θ±0.20, 17.30° 2θ±0.20, 18.07° 2θ±0.20, 18.58° 2θ±0.20, 19.13° 2θ±0.20, 19.39° 2θ±0.20, 19.56° 2θ±0.20, 20.95° 2θ±0.20, 21.64° 2θ±0.20, 22.18° 2θ±0.20, 22.45° 2θ±0.20, 23.03° 2θ±0.20, 23.38° 2θ±0.20, 23.98° 2θ±0.20, 24.74° 2θ±0.20, 24.95° 2θ±0.20, 25.19° 2θ±0.20, 25.71° 2θ±0.20, 26.08° 2θ±0.20, 26.47° 2θ±0.20, 27.28° 2θ±0.20, 27.81° 2θ±0.20, 28.41° 2θ±0.20, 28.80° 2θ±0.20, 30.13° 2θ±0.20, 30.66° 2θ±0.20, 31.90° 2θ±0.20, 32.16° 2θ±0.20, 32.57° 2θ±0.20, 33.37° 2θ±0.20, 33.75° 2θ±0.20, 34.77° 2θ±0.20, 35.29° 2θ±0.20, 36.25° 2θ±0.20, and 36.80° 2θ±0.20.

In another embodiment the Gentisate Form A may be characterized by an X-ray powder diffraction spectrum substantially as depicted in FIG. 10.

In another embodiment the acid is benzoic acid. The crystalline form is Benzoate Form A, for example as characterized by an X-ray powder diffraction spectrum substantially as depicted in FIG. 16.

In another embodiment the acid is fumaric acid. The crystalline form is Fumarate Form A, for example as characterized by an X-ray powder diffraction spectrum substantially as depicted in FIG. 19.

In another embodiment the acid is tartaric acid. The crystalline form is Tartrate Form A, for example as characterized by an X-ray powder diffraction spectrum substantially as depicted in FIG. 23.

In some embodiments the crystalline form may be stable after storage at 25° C., 40° C., or 70° C. for one day, one week, two weeks, one month, two months, three months, four months, five months, 6 months or at least one year.

The crystalline form may be more stable in water or saline compared to psilocin base in water or saline. For example, the crystalline form is stable for one day, one week, two weeks, one month, two months, three months, four months, five months, six months or at least one year during storage at 25° C., 40° C., or 70° C.

In some embodiments less 10% of the crystalline form degrades over a 36 hour period.

The solubility of the crystalline form may be at least about 0.25 mg/mL to at least about 10 mg/mL in water or saline.

In a second aspect there is provided a method of producing the crystalline form comprising the steps of:
a) reacting psilocin with the acid in a solvent, and
b) drying the resultant product of step a).

In a third aspect there is provided a pharmaceutical composition comprising the crystalline form of the first aspect. The pharmaceutical composition may be formulated for oral, subcutaneous, intravenous, or intramuscular administration, intravenous administration.

In a fourth aspect there is provided a method of treating or preventing a disease or condition in a subject comprising administering to the subject the crystalline form of the first aspect or the pharmaceutical composition of the third aspect.

In a fifth aspect there is provided use of the crystalline form f the first aspect or the pharmaceutical composition of the third aspect in the manufacture of a medicament for treating or preventing a disease or condition.

In a sixth aspect there is provided a crystalline form of the first aspect or the pharmaceutical composition of the third aspect for use in treating or preventing a disease or condition in a subject.

### BRIEF DESCRIPTION OF THE FIGURES

FIG. 1 shows an XRPD pattern of psilocin free base.

FIG. 2 shows an indexing solution for psilocin free base with the following characteristics:

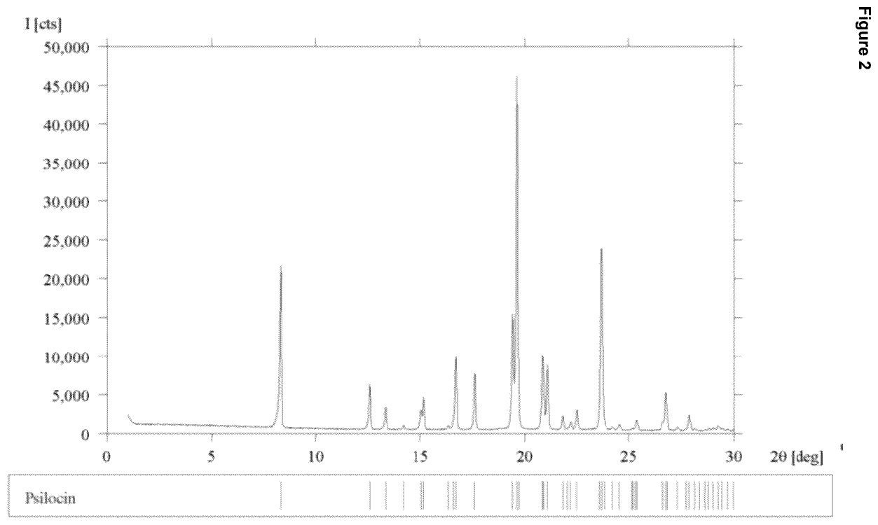

| | Psilocin free base |
|---|---|
| Bravais Type | Primitive monoclinic |
| a [Å] | 10.6000 |
| b [Å] | 8.518 |
| c [Å] | 12.478 |
| α [deg] | 90 |
| β [deg] | 90.99 |
| γ [deg] | 90 |
| Volume [Å³/cell] | 1,126.5 |
| Chiral contents ? | Not specified |
| Extinction Symbol | P 1 2₁/c 1 |
| Space Groups(s) | P2₁/c(14) |

FIG. 3 shows an ¹H NMR spectrum of psilocin free base.

FIG. 4 shows an XRPD pattern of psilocin besylate Form A.

FIG. 5 shows an indexing solution for psilocin besylate Form A with the following characteristics:

| | Psilocin besylate Form A |
|---|---|
| Bravais Type | Primitive orthorhombic |
| a [Å] | 9.014 |
| b [Å] | 17.213 |
| c [Å] | 22.944 |
| α [deg] | 90 |
| β [deg] | 90 |
| γ [deg] | 90 |
| Volume [Å³/cell] | 3,559.9 |
| Chiral contents ? | Not specified |
| Extinction Symbol | P b c a |
| Space Groups(s) | Pbca(61) |

FIG. 6 shows an ¹H NMR spectra of psilocin besylate Form A (middle), including reference spectra for psilocin free base (top) and benzenesulfonic acid (bottom).

FIG. 7 shows an XRPD pattern of psilocin butyrate Form A.

FIG. 8 shows an indexing solution for psilocin butyrate Form A with the following characteristics:

| | Psilocin butyrate Form A |
|---|---|
| Bravais Type | Primitive monoclinic |
| a [Å] | 9.015 |
| b [Å] | 11.515 |
| c [Å] | 16.842 |
| α [deg] | 90 |
| β [deg] | 100.33 |
| γ [deg] | 90 |
| Volume [Å³/cell] | 1720.2 |
| Chiral contents ? | Not specified |
| Extinction Symbol | P 1 2₁/c 1 |
| Space Groups(s) | P2₁/c(14) |

FIG. 9 shows an ¹H NMR spectra of psilocin butyrate Form A (bottom), including reference spectrum for psilocin free base (top).

FIG. 10 shows an XRPD overlay of psilocin gentisate Form A, including Gentisate Form A, RR (Reaction Ratio) 2:1 mol/mol, from EtOAc/2-8° C. (top); Gentisate Form A, post-dried, vacuum/RT/1 d (middle); and Gentisate Form A, preparation for additional materials (bottom).

FIG. 11 shows an indexing solution for psilocin gentisate Form A with the following characteristics:

| | Psilocin gentisate Form A |
|---|---|
| Bravais Type | Triclinic |
| a [Å] | 8.324 |
| b [Å] | 11.124 |
| c [Å] | 12.510 |
| α [deg] | 65.93 |
| β [deg] | 86.43 |
| γ [deg] | 74.82 |
| Volume [Å³/cell] | 1,019.5 |
| Chiral contents ? | Not specified |
| Extinction Symbol | P - |
| Space Groups(s) | P1 (1), P1̅(2) |

FIG. 12 shows an ¹H NMR spectra of psilocin gentisate Form A before drying (top middle) and after drying (bottom middle), including reference spectra for psilocin free base (top) and gentisic acid (bottom).

FIG. 13 shows an XRPD pattern of psilocin acetate Form A.

FIG. 14 shows an indexing solution for psilocin acetate Form A with the following characteristics:

| | Psilocin acetate Form A |
|---|---|
| Bravais Type | Primitive monoclinic |
| a [Å] | 10.279 |
| b [Å] | 9.472 |
| c [Å] | 15.893 |
| α [deg] | 90 |
| β [deg] | 102.6 |
| γ [deg] | 90 |
| Volume [Å³/cell] | 1,510.1 |
| Chiral contents ? | Not specified |
| Extinction Symbol | P 1 2₁/c 1 |
| Space Groups(s) | P2₁/c(14) |

FIG. 15 shows an ¹H NMR spectrum of psilocin acetate Form A (middle), including reference spectra for psilocin free base (top) and acetic acid (bottom).

FIG. 16 shows an XRPD overlay of psilocin benzoate Form A.

FIG. 17 shows an indexing solution for psilocin benzoate Form A with the following characteristics:

| | Psilocin benzoate Form A |
|---|---|
| Bravais Type | Primitive monoclinic |
| a [Å] | 9.623 |
| b [Å] | 11.528 |
| c [Å] | 16.849 |
| α [deg] | 90 |
| β [deg] | 106.25 |
| γ [deg] | 90 |
| Volume [Å³/cell] | 1,794.7 |
| Chiral contents ? | Not specified |
| Extinction Symbol | P 1 2₁/c 1 |
| Space Groups(s) | P2₁/c(14) |

FIG. 18 shows an ¹H NMR spectra of psilocin benzoate Form A before drying (top middle) and after drying (bottom middle), including reference spectra for psilocin free base (top) and benzoic acid (bottom).

FIG. 19 shows an XRPD overlay of psilocin fumarates Form A and Form B, including psilocin fumarate Form A, RR 1:1 mol/mol, from acetone/2-8° C. (top); psilocin fumarate Form B+2nd phase(s), from drying psilocin fumarate Form A (middle) and psilocin fumarate Form B, RR 1:1 mol/mol, from IPA and drying (bottom).

FIG. 20 shows an indexing solution for psilocin fumarate Form A with the following characteristics:

| | Psilocin fumarate Form A |
|---|---|
| Bravais Type | Primitive monoclinic |
| a [Å] | 11.087 |
| b [Å] | 10.453 |
| c [Å] | 16.198 |
| α [deg] | 90 |
| β [deg] | 110.01 |
| γ [deg] | 90 |
| Volume [Å³/cell] | 1,763.9 |
| Chiral contents ? | Not specified |
| Extinction Symbol | P 1 2₁/c 1 |
| Space Groups(s) | P2₁/c(14) |

FIG. 21 shows an indexing solution for psilocin fumarate Form B with the following characteristics:

| | Psilocin fumarate Form B |
|---|---|
| Bravais Type | Triclinic |
| a [Å] | 8.108 |
| b [Å] | 9.379 |
| c [Å] | 9.392 |
| α [deg] | 100.69 |
| β [deg] | 96.42 |
| γ [deg] | 101.94 |
| Volume [Å³/cell] | 678.2 |
| Chiral contents ? | Not specified |
| Extinction Symbol | P - |
| Space Groups(s) | P1 (1), P1̅(2) |

FIG. 22 shows an ¹H NMR spectra of psilocin fumarate Form A and psilocin fumarate Form B, including reference spectrum for psilocin free base (top); psilocin fumarate Form A (top middle); psilocin fumarate Form B (w/ minor 2nd phase) (bottom middle); and reference spectrum for fumaric acid (bottom).

FIG. 23 shows an XRPD pattern of psilocin tartrate Form A.

FIG. 24 shows an indexing solution for psilocin tartrate Form A with the following characteristics:

| | Psilocin tartrate Form A |
|---|---|
| Bravais Type | Primitive orthorhombic |
| a [Å] | 7.591 |
| b [Å] | 8.244 |
| c [Å] | 26.413 |
| α [deg] | 90 |
| β [deg] | 90 |
| γ [deg] | 90 |
| Volume [Å³/cell] | 1652.9 |
| Chiral contents ? | Chiral |
| Extinction Symbol | P 2₁ 2₁ 2₁ |
| Space Groups(s) | P2₁2₁2₁ (19) |

FIG. 25 shows an ¹H NMR spectrum of psilocin tartrate Form A (middle), including reference spectra for psilocin free base (top) and tartaric acid (bottom).

FIG. 26 shows the stability over time of psilocin besylate, psilocin butyrate and psilocin gentisate compared to psilocin in saline solution. This graph is based on psilocin peak area over time

FIG. 27 shows the stability over time of psilocin besylate, psilocin butyrate and psilocin gentisate compared to psilocin in saline solution. This graph shows psilocin peak area as a percentage over time.

### DEFINITIONS

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps. The term “consisting of” means “consisting only of”, that is, including and limited to the stated element(s), integer(s) or step(s), and excluding any other element(s), integer(s) or step(s). The term “consisting essentially of” means the inclusion of the stated element(s), integer(s) or step(s), but other element(s), integer(s) or step(s) that do not materially alter or contribute to the working of the invention may also be included.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms “a” and “an” are used to refer to one or more than one (i.e., at least one) of the grammatical object of the article. By way of example, reference to “an element” means one element, or more than one element.

In the context of the present specification the term “about” means that reference to a figure or value is not to be taken as an absolute figure or value but includes margins of variation above or below the figure or value in line with what a skilled person would understand according to the art, including within typical margins of error or instrument limitation. In other words, use of the term “about” is understood to refer to a range or approximation that a person or skilled in the art would consider to be equivalent to a recited value in the context of achieving the same function or result.

The term “pharmaceutically acceptable salt” refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19. For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art. The salts can be prepared in situ during the final isolation and purification of the compounds of the invention, or separately by reacting the free base function with a suitable organic acid. Suitable pharmaceutically acceptable acid addition salts of the compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, methanesulfonic, salicylic, p-hydroxybenzoic, phenylacetic, mandelic, ambonic, pamoic, pantothenic, sulfanilic, cyclohexylaminosulfonic, stearic, algenic, β-hydroxybutyric, galactaric, fumaric and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, suitable pharmaceutically acceptable base addition salts of the compounds of the present invention include organic salts made from N,N′-dibenzylethylenediamine, chloroprocaine, choline, diethanolamine, ethylenediamine, meglumine (N methylglucamine), procaine, ammonium salts, quaternary salts such as tetramethylammonium salt, amino acid addition salts such as salts with glycine and arginine. In the case of compounds that are solids, it will be understood by those skilled in the art that the inventive compounds, agents and salts may exist in different crystalline or polymorphic forms, all of which are intended to be within the scope of the present invention and specified formulae.

The terms “treating”, “treatment” and “therapy” are used herein to refer to curative therapy, prophylactic therapy, palliative therapy and preventative therapy. Thus, in the context of the present disclosure the term “treating” encompasses curing, ameliorating or tempering the severity of a medical condition or one or more of its associated symptoms.

The terms “therapeutically effective amount” or “pharmacologically effective amount” or “effective amount” refer to an amount of an agent sufficient to produce a desired therapeutic or pharmacological effect in the subject being treated. The terms are synonymous and are intended to qualify the amount of each agent that will achieve the goal of improvement in disease severity and/or the frequency of incidence over treatment of each agent by itself while preferably avoiding or minimising adverse side effects, including side effects typically associated with other therapies. Those skilled in the art can determine an effective dose using information and routine methods known in the art.

A “pharmaceutical carrier, diluent or excipient” includes, but is not limited to, any physiological buffered (i.e., about pH 6.0 to 7.4) medium comprising a suitable water-soluble organic carrier, conventional solvents, dispersion media, fillers, solid carriers, coatings, antibacterial and antifungal agents, isotonic and absorption delaying agents. Suitable water-soluble organic carriers include, but are not limited to, saline, dextrose, corn oil, dimethylsulfoxide, and gelatin capsules. Other conventional additives include lactose, mannitol, corn starch, potato starch, binders such as microcrystalline cellulose, cellulose derivatives such as hydroxypropylmethylcellulose, acacia, gelatins, disintegrators such as sodium carboxymethylcellulose, and lubricants such as talc or magnesium stearate.

“Subject” includes any human or non-human mammal. Thus, in addition to being useful for human treatment, the compounds of the present invention may also be useful for veterinary treatment of mammals, including companion animals and farm animals, such as, but not limited to dogs, cats, horses, cows, sheep, and pigs. In preferred embodiments the subject is a human.

In the context of this specification the term “administering” and variations of that term including “administer” and “administration”, includes contacting, applying, delivering or providing a compound or composition of the invention to a subject by any appropriate means.

### Abbreviations and Glossary

“HPLC” refers to High-performance liquid chromatography

“NMR” refers to Nuclear magnetic resonance spectroscopy

“PLM” refers to Polarized laser microscopy

“XRPD” refers to X-ray powder diffraction

“FE” refers to Fast evaporation

“ACN” refers to Acetonitrile

“DMSO” refers to Dimethyl sulfoxide

“EtOAc” refers to Ethyl acetate

“EtOH” refers to Ethanol

“GRAS” refers to Generally Regarded as Safe

“HCl” refers to Hydrochloric acid

“H₂O” refers to Water

“H₃PO₄” refers to Phosphoric acid

“IPA” refers to Isopropyl alcohol

“MeOH” refers to Methanol

“MTBE” refers to Methyl tert-butyl ether

“THF” refers to Tetrahydrofuran

“agg.” refers to aggregates/agglomerates

“B/E” refers to birefringence/extinction

“d” refers to day(s)

“h” refers to hour(s)

“IV” refers to intravenous

“LIMS” refers to laboratory information management system

“mol” refers to mole(s)

“min” refers to minute(s)

“N₂” refers to nitrogen

“RR” refers to reaction ratio

“RT” refers to room temperature

“w/” refers to with

“API Material X” refers to material confirmed to contain the API but of unknown crystalline form.

“API Form X” refers to material confirmed to contain an API and demonstrated to be constituted of a single crystalline form.

“API salt/cocrystal Material X” refers to material confirmed to contain a salt or cocrystal of the API but of unknown crystalline form.

“API salt/cocrystal Form X” refers to material confirmed to contain a salt or cocrystal of an API and demonstrated to be constituted of a single crystalline form.

“Crystalline” refers to a substance that produces an XRPD pattern with sharp peaks (similar to instrumental peak widths) and weak diffuse scattering (relative to the peaks).

“Disordered crystalline” refers to a substance that produces an XRPD pattern with broad peaks (relative to instrumental peak widths) and/or strong diffuse scattering (relative to the peaks). Disordered materials may be:
microcrystalline
crystalline with large defect density
mixtures of crystalline and X-ray amorphous phases
   or a combination of the above. Additional analysis may differentiate among these options.

“Insufficient signal” refers to circumstances where insufficient signal above the expected background scattering was observed. This may indicate that the X-ray beam missed the sample and/or that the sample was of insufficient mass for analysis.

“Particle statistics artifacts” refers to the circumstances where particle size distribution contains a small number of large crystals which may lead to sharp spikes in the XRPD pattern.

“Preferred orientation artifacts” refers to circumstances where the particle morphology is prone to non-random orientation in the sample holder which may lead to subtle and/or dramatic changes in relative peak intensities.

“No peaks” refers to circumstances where no Bragg peaks are observed in the XRPD pattern. The absence of peaks may be due to an X-ray amorphous sample and/or insufficient signal.

“Single crystalline phase” refers to circumstances where an XRPD pattern is judged to contain evidence of a single crystalline phase if all the Bragg peaks can be indexed with a single unit cell.

“X-ray amorphous” refers to circumstances where diffuse scatter is present, but no evidence for Bragg peaks in an XRPD pattern. X-ray amorphous materials may be:
nano-crystalline
crystalline with a very large defect density
kinetic amorphous material
thermodynamic amorphous material
   or a combination of the above. Additional analysis may differentiate among these options.

### DETAILED DESCRIPTION

The invention will be described with reference to various specific and preferred embodiments and techniques. However, it should be understood that many variations and modification may be made while remaining within the spirit and scope of the invention.

In a preferred embodiment, the invention relates to a crystalline form of a pharmaceutically acceptable salt of psilocin (4-hydroxy-N,N-dimethyltryptamine), or a cocrystal thereof, wherein the cocrystal comprises a coformer. Preferably, the crystalline form is Besylate Form A, Butyrate Form A, or Gentisate Form A.

The crystalline forms described herein have a number of advantages compared to psilocin. These advantages may include one or more of the following:
Increased solubility.
Improved stability.
Reduced interindividual variability in plasma concentrations following administration
Self-preserved formula.
Improved method of manufacture.
Increased bioavailability.
Improved side-effect profile.

In some embodiments, the crystalline forms described herein may provide enhanced physical properties, such as solubility, dissolution rate, bioavailability, physical stability, chemical stability, flowability, fractability, or compressibility. In some embodiments, a given API may form different cocrystals with one or more different counter-molecules, and some of these cocrystals may exhibit enhanced solubility or stability.

In one embodiment, crystalline form of psilocin is in the form of a pharmaceutically acceptable salt. The pharmaceutically acceptable salt may be selected from any pharmaceutically acceptable salt known in the art. Preferably, the pharmaceutically acceptable salt is a base form of an acid. The acid may be selected from the group consisting of acetic acid, aconitic acid, ascorbic acid, benzenesulfonic acid, benzoic acid, butyric acid, citric acid, erythorbic acid, fumaric acid, gentisic acid, glutamic acid, glycolic acid, hydrochloric acid, maleic acid, phosphoric acid, pyrogluamic acid, sorbic acid, succinic acid, sulfuric acid and tartaric acid. Preferably, the acid is benzenesulfonic acid, butyric acid, gentisic acid, acetic acid, benzoic acid, fumaric acid, or tartaric acid. Even more preferably the acid is benzenesulfonic, butyric or gentisic acid.

In some embodiments, the crystalline form of a pharmaceutically acceptable salt of psilocin is a cocrystal comprising a coformer. The coformer may be any pharmaceutically acceptable coformer known in the art. Preferably, the coformer is arginine, acetylsalicylic acid, glucose, nicotinic acid, aconitic acid, glutamic acid, oxalic acid, adipic acid, glutaric acid, proline, 4-aminosalicylic acid, glycine, propyl gallate, ascorbic acid, glycolic acid, pyroglutamic acid, benzoic acid, hippuric acid, saccharin, camphoric acid, 1-hydroxy-2-naphthoic acid, salicylic acid, capric acid, ketoglutaric acid, sebacic acid, cinnamic acid, lysine, sodium lauryl sulfate, citric acid, magnesium bromide, sorbic acid, cyclamic acid, maleic acid, succinic acid, ethyl maltol, malic acid, tartaric acid, ethyl paraben, malonic acid, urea, fructose, maltol, vanillic acid, fumaric acid, mandelic acid, vanillin, gallic acid, methyl paraben, zinc chloride, gentisic acid, nicotinamide or ethyl acetate. Preferably, the coformer is selected from the group consisting of arginine, lysine, methyl paraben, nicotinamide and ethyl acetate. Preferably, the coformer is ethyl acetate.

In one embodiment, the present invention may be a crystalline form or an amorphous form or mixtures thereof (e.g., mixtures of crystal forms, or mixtures of crystal and amorphous forms), which comprises (a) psilocin or a pharmaceutically acceptable salt, solvate, hydrate, stereoisomer, prodrug, or clathrate thereof and (b) a coformer.

In one embodiment, provided herein is a crystal form comprising (a) psilocin or a pharmaceutically acceptable salt, solvate, hydrate, stereoisomer, prodrug, or clathrate thereof and (b) a coformer.

In one embodiment, provided herein is a cocrystal comprising (a) psilocin or a pharmaceutically acceptable salt, solvate, hydrate, stereoisomer, prodrug, or clathrate thereof and (b) a coformer.

In one embodiment, provided herein is an amorphous form comprising (a) psilocin or a pharmaceutically acceptable salt, solvate, hydrate, stereoisomer, prodrug, or clathrate thereof and (b) a coformer.

In one embodiment, provided herein is a mixture comprising (i) a cocrystal comprising (a) psilocin or a pharmaceutically acceptable salt, solvate, hydrate, stereoisomer, prodrug, or clathrate thereof and (b) a coformer; and (ii) a crystal form of psilocin or a pharmaceutically acceptable salt, solvate, hydrate, stereoisomer, prodrug, or clathrate thereof.

In one embodiment, provided herein is a mixture comprising (i) a cocrystal comprising (a) psilocin or a pharmaceutically acceptable salt, solvate, hydrate, stereoisomer, prodrug, or clathrate thereof and (b) a coformer; and (ii) an amorphous form of psilocin or a pharmaceutically acceptable salt, solvate, hydrate, stereoisomer, prodrug, or clathrate thereof.

In a preferred embodiment, the crystalline form is Besylate Form A. Preferably, the Besylate Form A has an XRPD (X-ray power diffraction) pattern having peaks at about 15.44° 2θ±0.20, 18.33° 2θ±0.20, and 25.41° 2θ±0.20. In another embodiment the XRPD (X-ray power diffraction) pattern has peaks at about 14.73° 2θ±0.20, 15.44° 2θ±0.20, 18.33° 2θ±0.20, 22.59° 2θ±0.20, and 25.41° 2θ±0.20. In another embodiment the XRPD (X-ray power diffraction) peaks at about 11.72° 2θ±0.20, 12.47° 2θ±0.20, 13.49° 2θ±0.20, 14.73° 2θ±0.20, 15.44° 2θ±0.20, 18.33° 2θ±0.20, 20.62° 2θ±0.20, 20.99° 2θ±0.20, 21.77° 2θ±0.20, 22.25° 2θ±0.20, 22.59° 2θ±0.20, 23.22° 2θ±0.20, 23.71° 2θ±0.20, 24.10° 2θ±0.20, and 25.41° 2θ±0.20.

In one embodiment the Besylate Form A has an XRPD (X-ray power diffraction) pattern having peaks at about 15.44° 2θ The crystalline form of claim 4, wherein the crystalline form is Besylate Form A and exhibits XRPD (X-ray power diffraction) peaks at about 7.69° 2θ±0.20, 10.26° 2θ±0.20, 10.96° 2θ±0.20, 11.72° 2θ±0.20, 12.47° 2θ±0.20, 12.84° 2θ±0.20, 13.49° 2θ±0.20, 14.73° 2θ±0.20, 15.44° 2θ±0.20, 16.18° 2θ±0.20, 18.33° 2θ±0.20, 19.04° 2θ±0.20, 19.68° 2θ±0.20, 20.62° 2θ±0.20, 20.99° 2θ±0.20, 21.77° 2θ±0.20, 22.25° 2θ±0.20, 22.59° 2θ±0.20, 23.22° 2θ±0.20, 23.71° 2θ±0.20, 24.10° 2θ±0.20, 25.15° 2θ±0.20, 25.41° 2θ±0.20, 25.65° 2θ±0.20, 26.29° 2θ±0.20, 26.76° 2θ±0.20, 27.72° 2θ±0.20, 27.99° 2θ±0.20, 28.67° 2θ±0.20, 28.93° 2θ±0.20, 29.63° 2θ±0.20, 30.43° 2θ±0.20, 30.76° 2θ±0.20, 31.15° 2θ±0.20, 31.77° 2θ±0.20, 32.13° 2θ±0.20, 32.94° 2θ±0.20, 33.65° 2θ±0.20, 34.94° 2θ±0.20, 35.69° 2θ±0.20, and 36.49° 2θ±0.20.

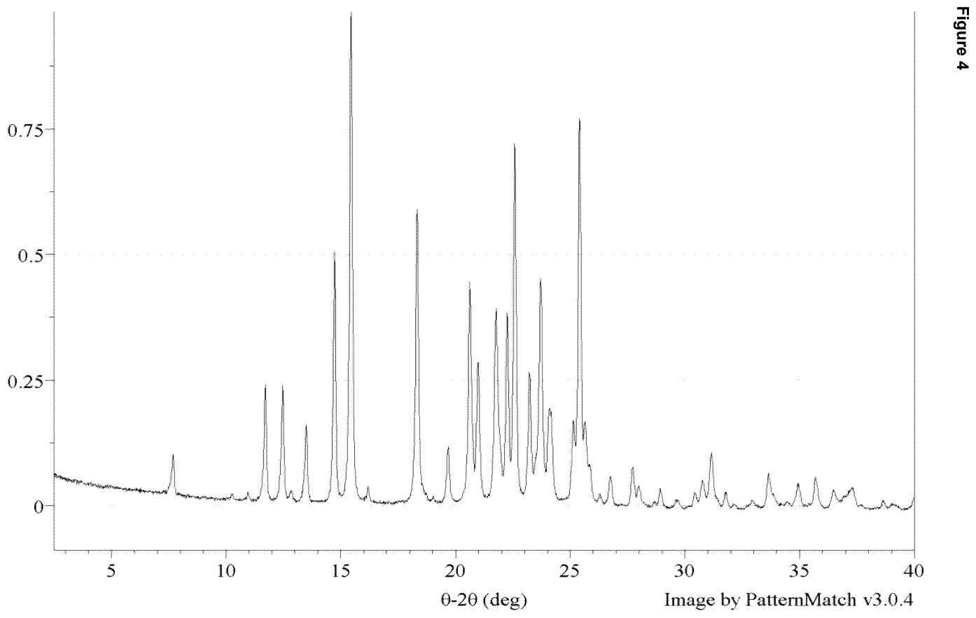

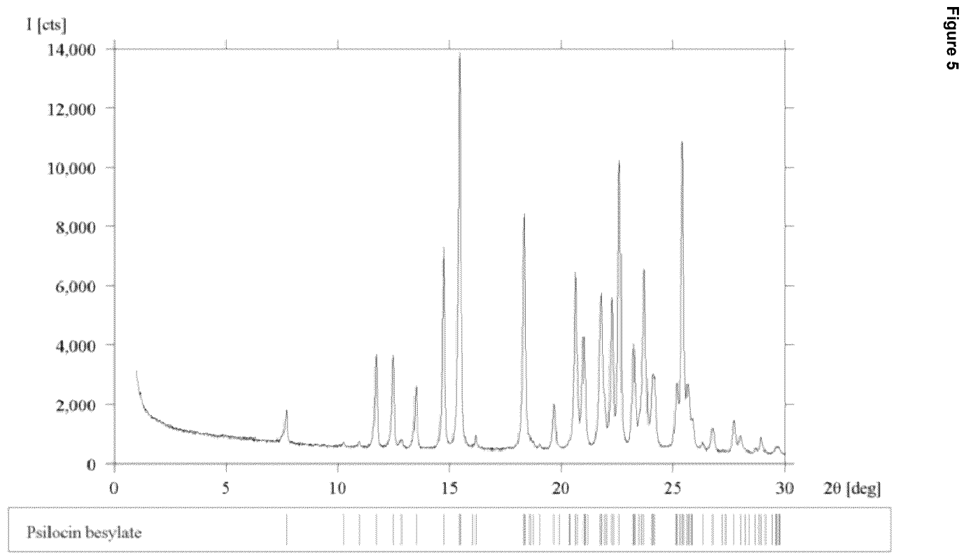

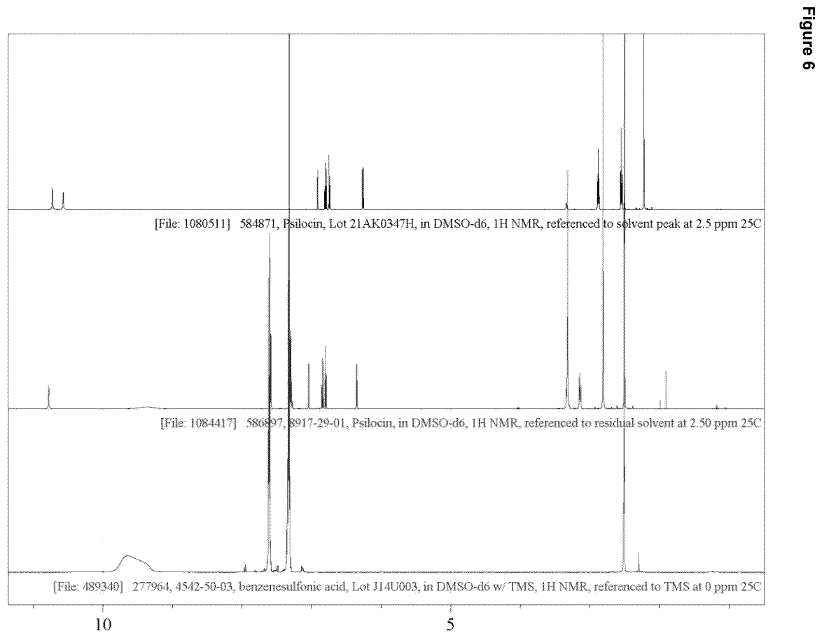

In one embodiment, the Besylate Form A has an XRPD pattern comprising peaks substantially or essentially the same as shown in FIG. 4 or 5. In one embodiment, the Besylate Form A has an 1H NMR spectrum comprising peaks substantially or essentially the same as shown in FIG. 6. The Besylate Form A may be solvated, hemi-solvated or unsolvated. In a preferred embodiment, the Besylate Form A is unsolvated.

In another preferred embodiment, the crystalline form is Butyrate Form A. Preferably, the Butyrate Form A has an XRPD pattern having peaks at about 13.24° 2θ±0.20, 15.34° 2θ±0.20, and 15.88° 2θ±0.20.

In another embodiment the Butyrate Form A has an XRPD pattern having peaks at about 13.24° 2θ±0.20, 15.34° 2θ±0.20, 15.88° 2θ±0.20, 16.28° 2θ±0.20, 20.95° 2θ±0.20, and 27.79° 2θ±0.20.

In another embodiment the Butyrate Form A has an XRPD pattern having peaks at about 9.33° 2θ±0.20, 9.96° 2θ±0.20, 10.66° 2θ±0.20, 13.24° 2θ±0.20, 15.34° 2θ±0.20, 15.88° 2θ±0.20, 16.28° 2θ±0.20, 17.80° 2θ±0.20, 20.95° 2θ±0.20, 21.98° 2θ±0.20, 22.32° 2θ±0.20, 23.31° 2θ±0.20, 24.61° 2θ±0.20, and 27.79° 2θ±0.20.

In a further embodiment the Butyrate Form A has an XRPD pattern having peaks at about at about 9.33° 2θ±0.20, 9.96° 2θ±0.20, 10.66° 2θ±0.20, 12.96° 2θ±0.20, 13.24° 2θ±0.20, 14.36° 2θ±0.20, 15.34° 2θ±0.20, 15.88° 2θ±0.20, 16.28° 2θ±0.20, 17.80° 2θ±0.20, 18.36° 2θ±0.20, 18.75° 2θ±0.20, 18.97° 2θ±0.20, 19.63° 2θ±0.20, 20.01° 2θ±0.20, 20.35° 2θ±0.20, 20.95° 2θ±0.20, 21.44° 2θ±0.20, 21.98° 2θ±0.20, 22.32° 2θ±0.20, 22.80° 2θ±0.20, 23.31° 2θ±0.20, 23.77° 2θ±0.20, 24.41° 2θ±0.20, 24.61° 2θ±0.20, 25.46° 2θ±0.20, 25.60° 2θ±0.20, 26.22° 2θ±0.20, 26.67° 2θ±0.20, 27.79° 2θ±0.20, and 29.07° 2θ±0.20.

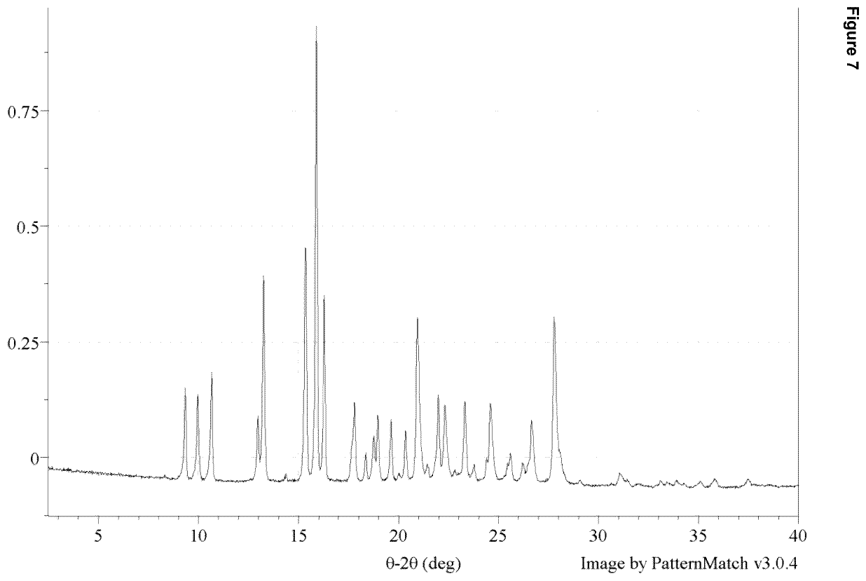

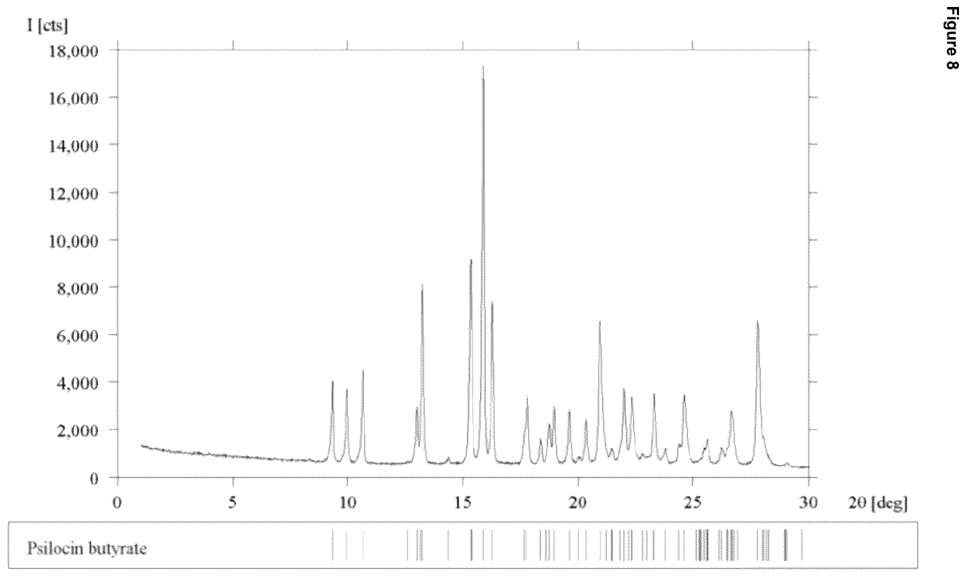

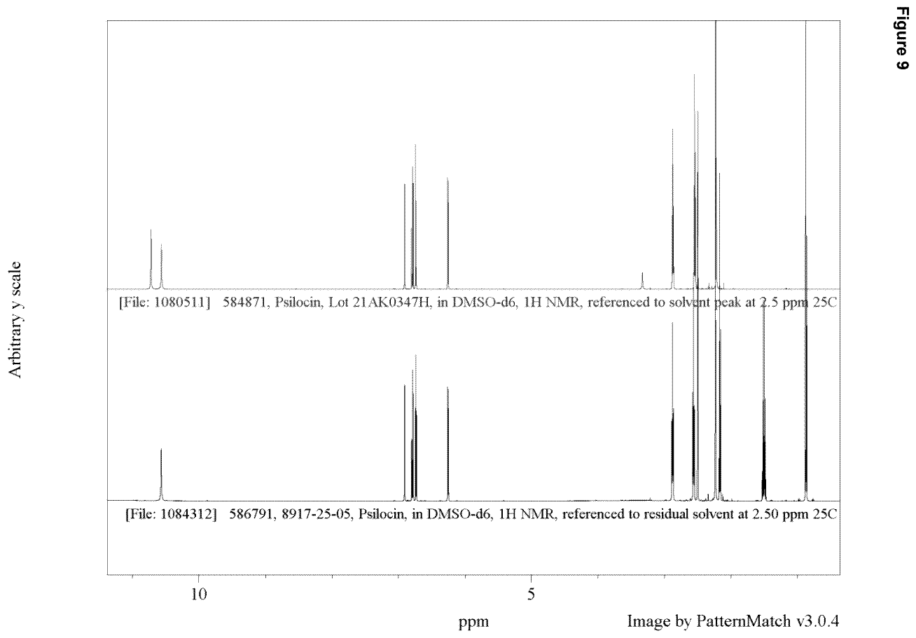

In one embodiment, the Butyrate Form A has an XRPD pattern comprising peaks substantially or essentially the same as shown in FIG. 7 or 8. In one embodiment, the Butyrate Form A has an ¹H NMR spectrum comprising peaks substantially or essentially the same as shown in FIG. 9. The Butyrate Form A may be solvated, hemi-solvated or unsolvated. Preferably, the Butyrate Form A is unsolvated.

In another preferred embodiment, the crystalline form is Gentisate Form A. Preferably, the Gentisate Form A has an XRPD pattern having peaks at about 15.80° 2θ±0.20, 16.51° 2θ±0.20, and 23.98° 2θ±0.20.

In another embodiment the Gentisate Form A has an XRPD pattern having peaks at about 15.52° 2θ±0.20, 15.80° 2θ±0.20, 16.51° 2θ±0.20, 23.98° 2θ±0.20, and 24.74° 2θ±0.20. In another embodiment the Gentisate Form A has an XRPD pattern having peaks at about at about 12.77° 2θ±0.20, 14.08° 2θ±0.20, 15.52° 2θ±0.20, 15.80° 2θ±0.20, 15.98±0.20, 16.51° 2θ±0.20, 17.30° 2θ±0.20, 18.58° 2θ±0.20, 20.95° 2θ±0.20, 21.64° 2θ±0.20, 23.38° 2θ±0.20, 23.98° 2θ±0.20, 24.74° 2θ±0.20, 25.19° 2θ±0.20, 27.81° 2θ±0.20, 28.41° 2θ±0.20, and 28.80° 2θ±0.20.

In another embodiment Gentisate Form A has an XRPD pattern having peaks at about 7.74° 2θ±0.20, 9.01° 2θ±0.20, 11.01° 2θ±0.20, 12.29° 2θ±0.20, 12.77° 2θ±0.20, 13.15° 2θ±0.20, 13.80° 2θ±0.20, 14.08° 2θ±0.20, 15.52° 2θ±0.20, 15.80° 2θ±0.20, 15.98° 2θ±0.20, 16.11° 2θ±0.20, 16.51° 2θ±0.20, 17.30° 2θ±0.20, 18.07° 2θ±0.20, 18.58° 2θ±0.20, 19.13° 2θ±0.20, 19.39° 2θ±0.20, 19.56° 2θ±0.20, 20.95° 2θ±0.20, 21.64° 2θ±0.20, 22.18° 2θ±0.20, 22.45° 2θ±0.20, 23.03° 2θ±0.20, 23.38° 2θ±0.20, 23.98° 2θ±0.20, 24.74° 2θ±0.20, 24.95° 2θ±0.20, 25.19° 2θ±0.20, 25.71° 2θ±0.20, 26.08° 2θ±0.20, 26.47° 2θ±0.20, 27.28° 2θ±0.20, 27.81° 2θ±0.20, 28.41° 2θ±0.20, 28.80° 2θ±0.20, 30.13° 2θ±0.20, 30.66° 2θ±0.20, 31.90° 2θ±0.20, 32.16° 2θ±0.20, 32.57° 2θ±0.20, 33.37° 2θ±0.20, 33.75° 2θ±0.20, 34.77° 2θ+0.20, 35.29° 2θ±0.20, 36.25° 2θ±0.20, and 36.80° 2θ±0.20.

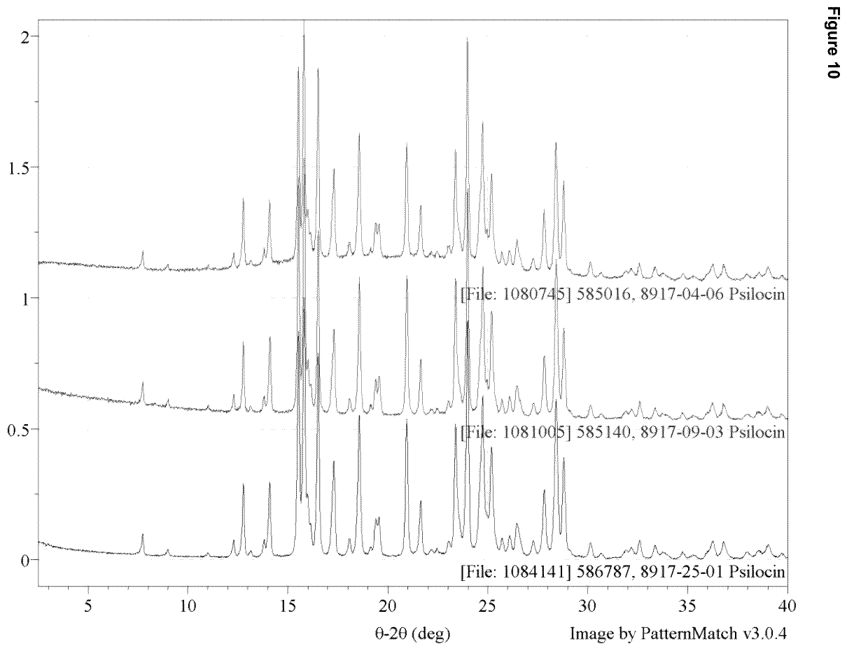

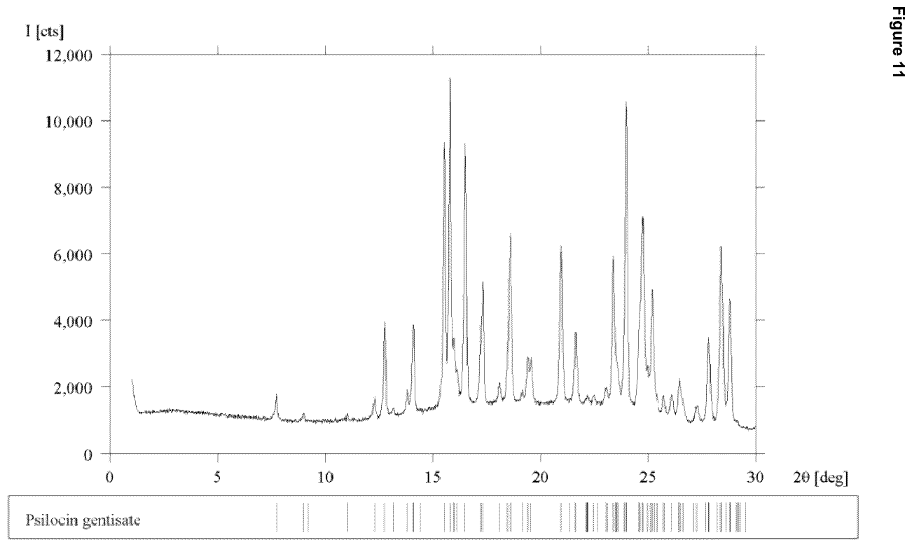

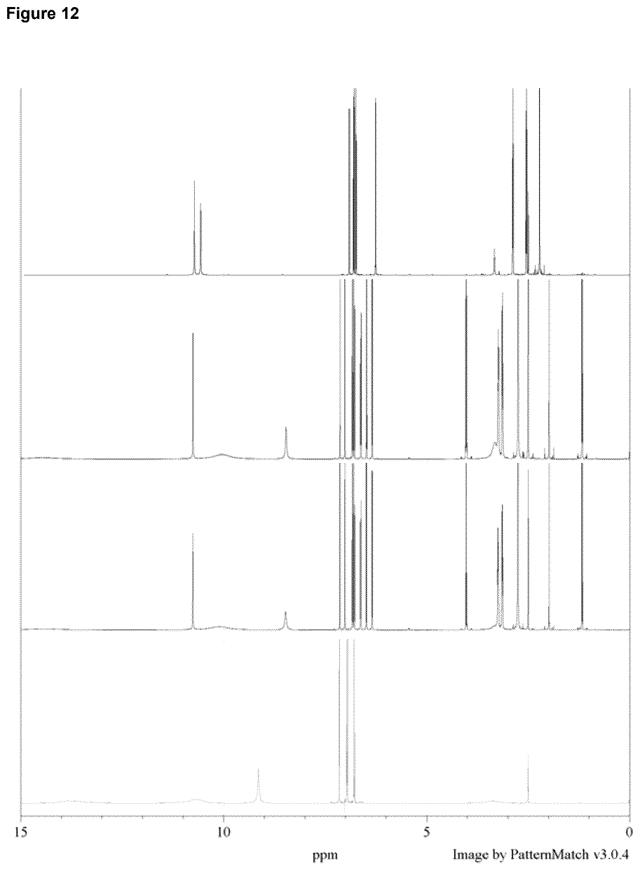

In one embodiment, the Gentisate Form A has an XRPD pattern comprising peaks substantially or essentially the same as shown in FIG. 10 or 11. In one embodiment, the Gentisate Form A has an ¹H NMR spectrum comprising peaks substantially or essentially the same as shown in FIG. 12. The Gentisate Form A may be solvated, hemi-solvated or unsolvated. Preferably, the Gentisate Form A is hemi-solvated or unsolvated.

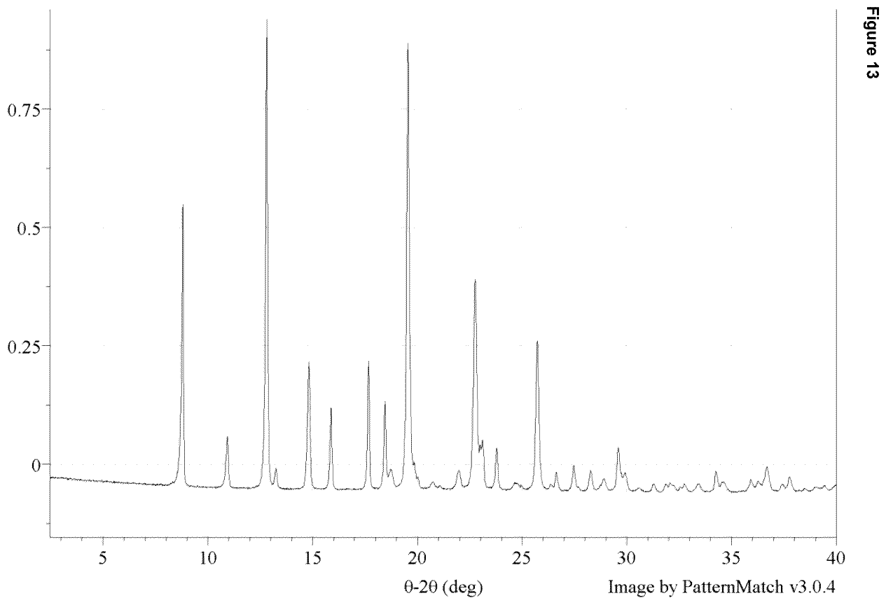

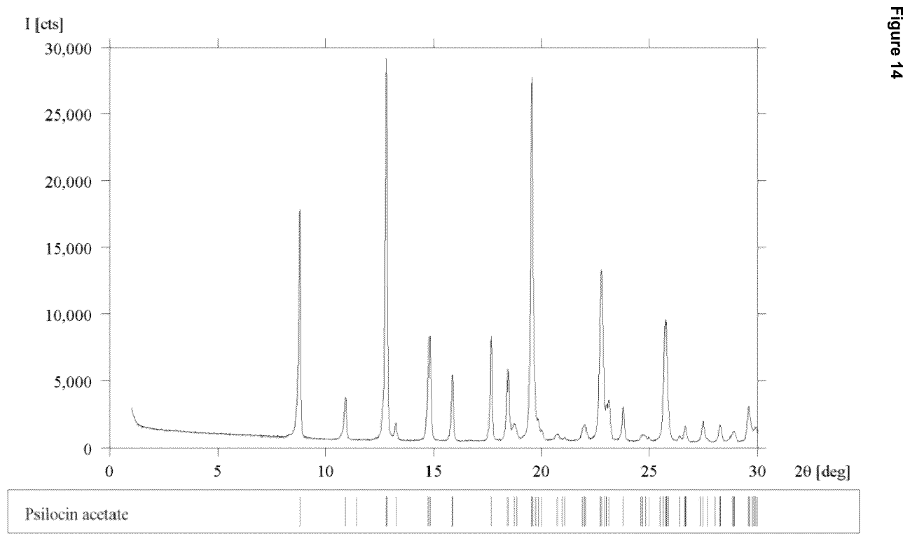

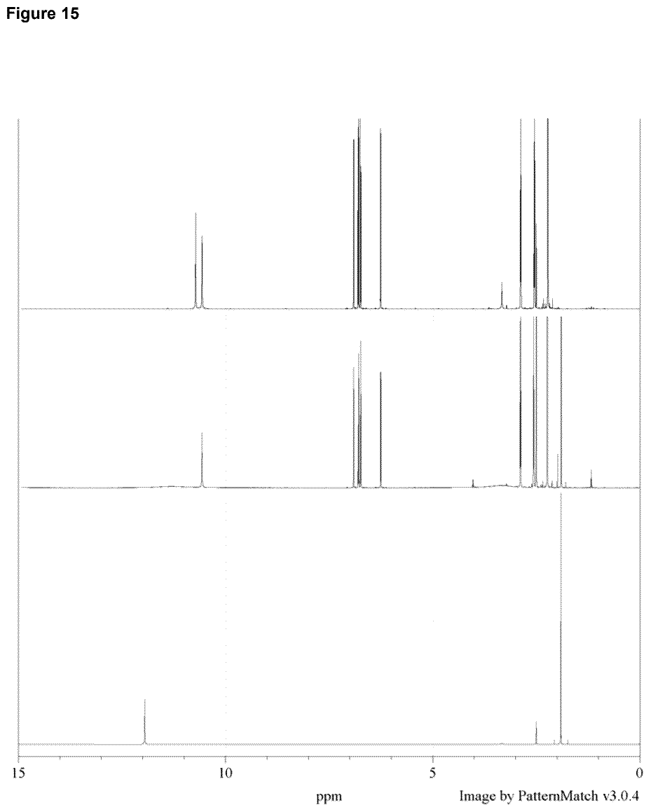

In another embodiment, the crystalline form is Acetate Form A. Preferably, the Acetate Form A has an XRPD pattern having comprising peaks substantially or essentially the same as shown in FIG. 13 or 14. In one embodiment, the Acetate Form A has an ¹H NMR spectrum comprising peaks substantially or essentially the same as shown in FIG. 15. The Acetate Form A may be solvated, hemi-solvated or unsolvated.

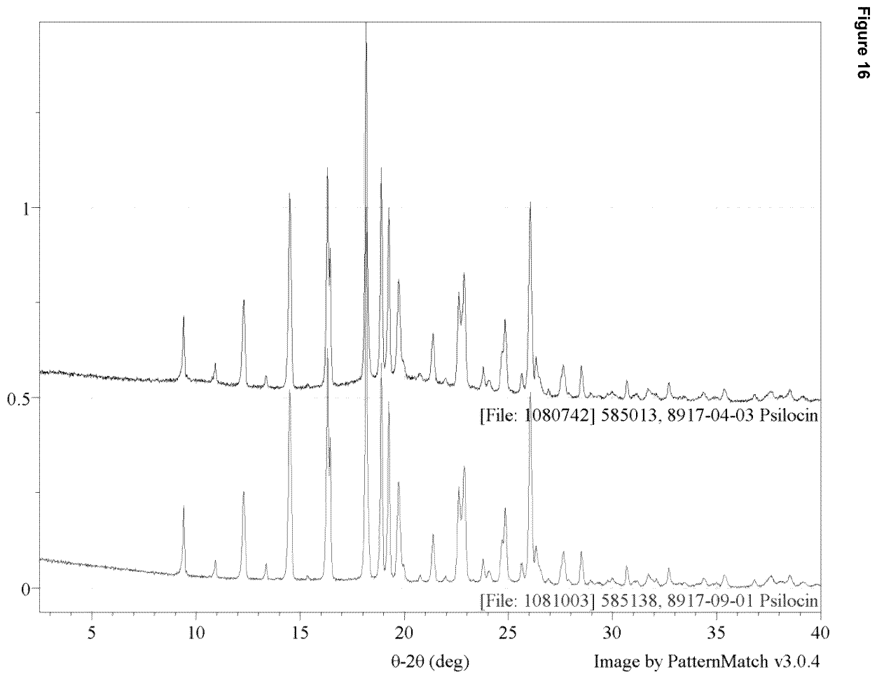

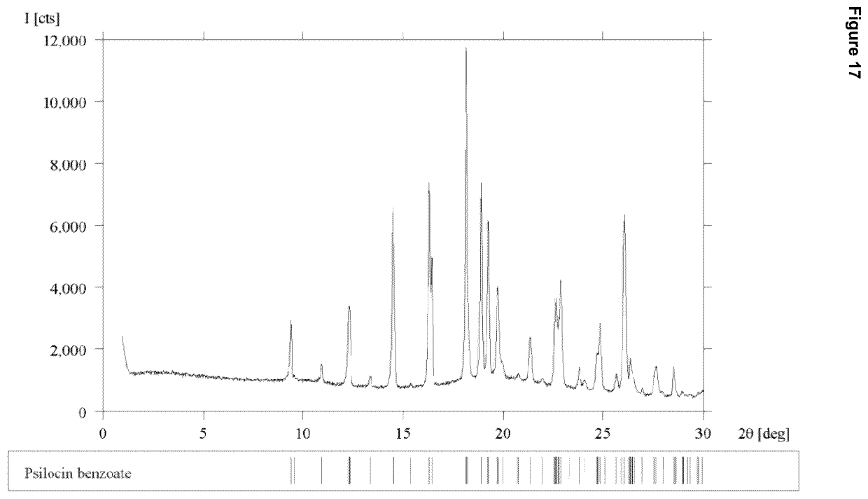

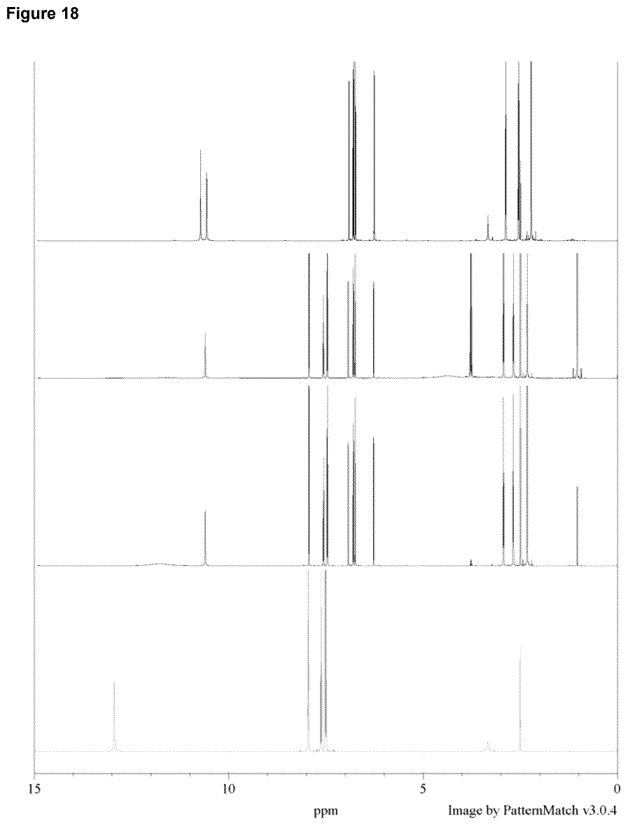

In another embodiment, the crystalline form is Benzoate Form A. Preferably, the Benzoate Form A has an XRPD pattern comprising peaks substantially or essentially the same as shown in FIG. 16 or 17. In one embodiment, the Benzoate Form A has an ¹H NMR spectrum comprising peaks substantially or essentially the same as shown in FIG. 18. The Benzoate Form A may be solvated, hemi-solvated or unsolvated.

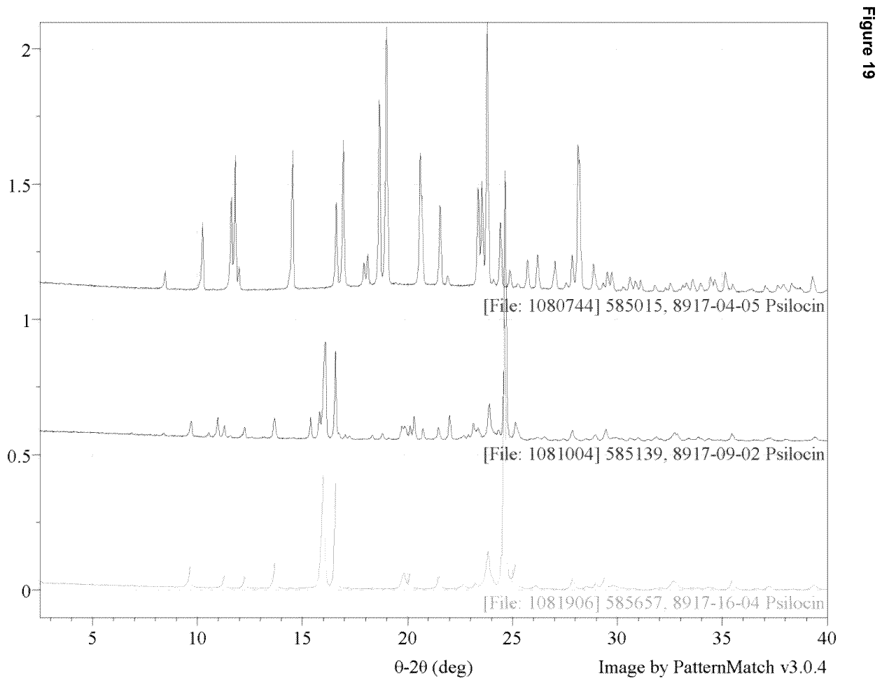

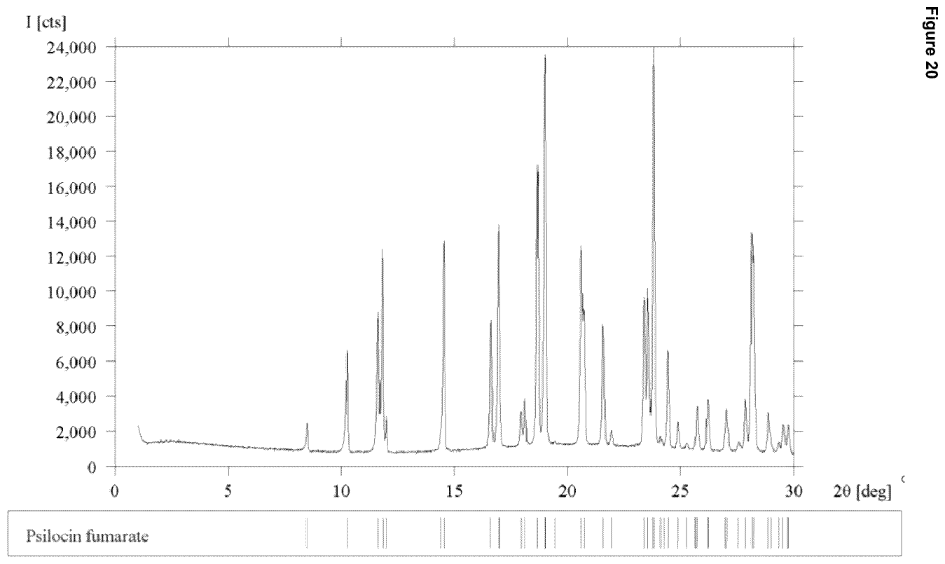

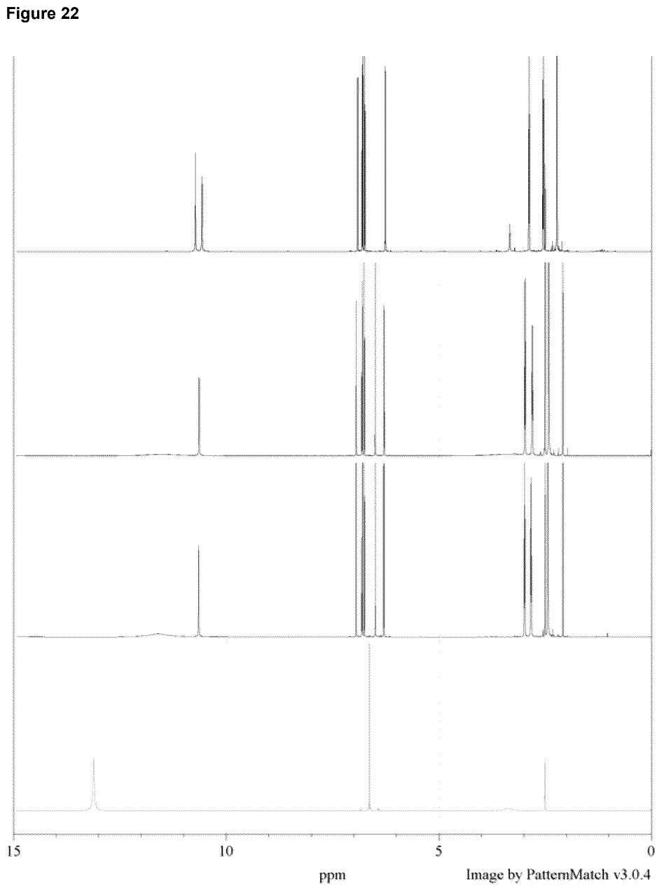

In another embodiment, the crystalline form is Fumarate Form A. Preferably, the Fumarate Form A has an XRPD pattern comprising peaks substantially or essentially the same as shown in FIG. 19 or 20. In one embodiment, the Fumarate Form A has an ¹H NMR spectrum comprising peaks substantially or essentially the same as shown in FIG. 22. The Fumarate Form A may be solvated, hemi-solvated or unsolvated.

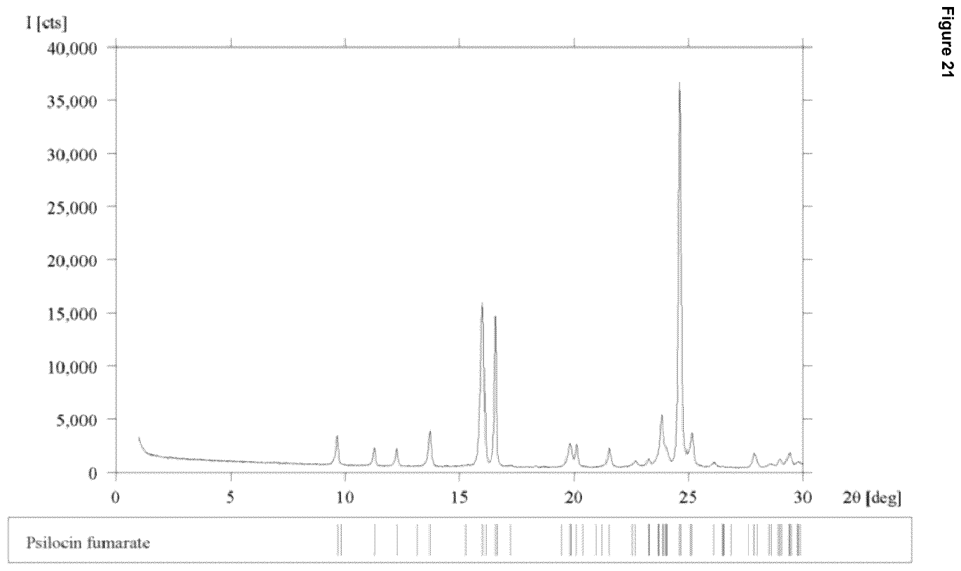

In another embodiment, the crystalline form is Fumarate Form B. Preferably, the Fumarate Form B has an XRPD pattern comprising peaks substantially or essentially the same as shown in FIG. 19 or 21. In one embodiment, the Fumarate Form B has an ¹H NMR spectrum comprising peaks substantially or essentially the same as shown in FIG. 22. The Fumarate Form B may be solvated, hemi-solvated or unsolvated.

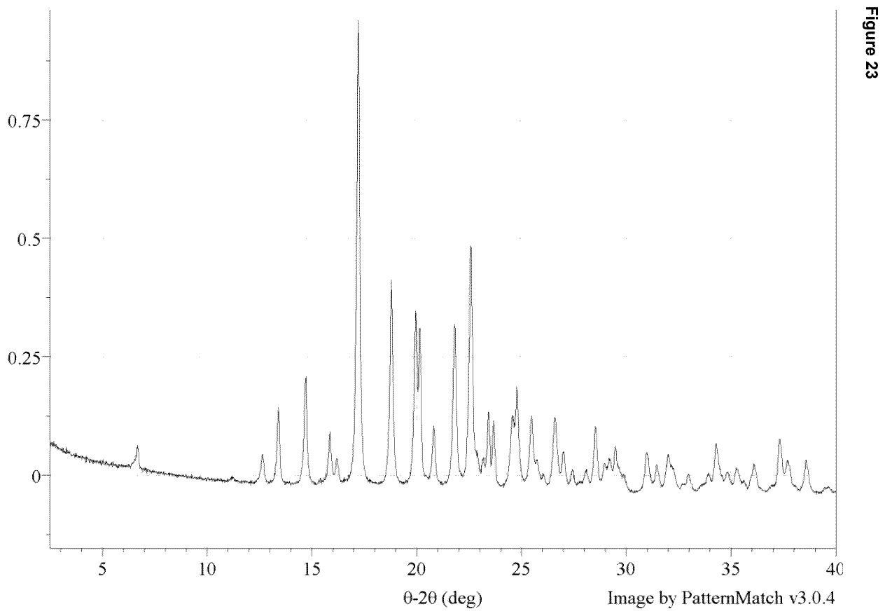

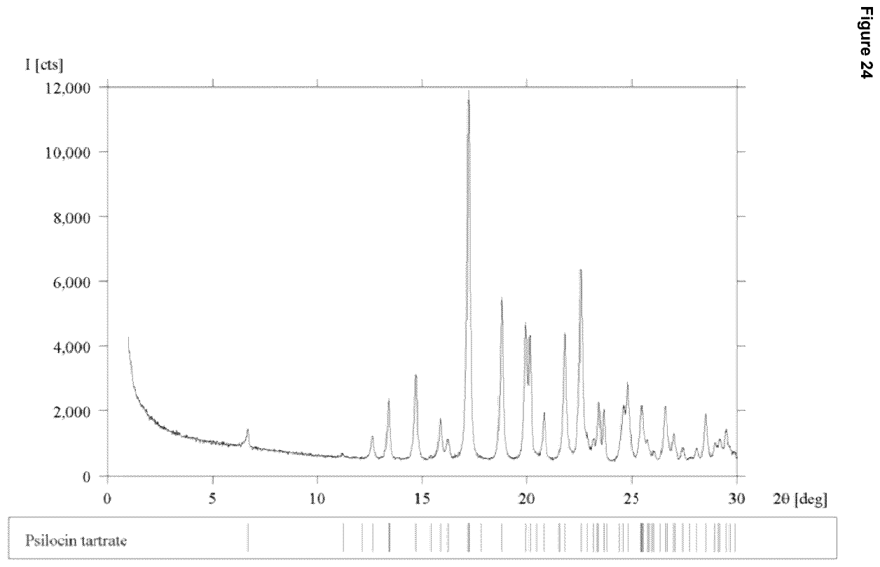

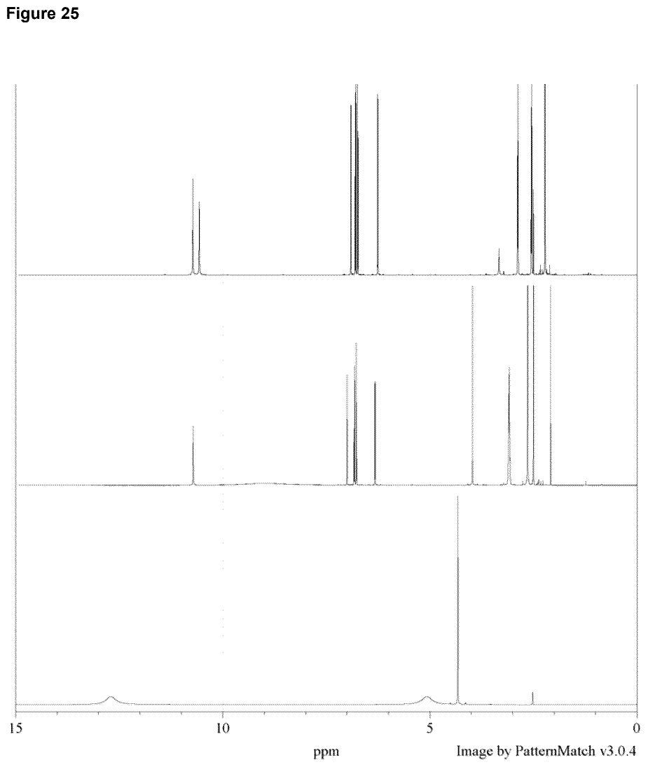

In another embodiment, the crystalline form is Tartrate Form A. Preferably, the Tartrate Form A has an XRPD pattern comprising peaks substantially or essentially the same as shown in FIG. 23 or 24. In one embodiment, the Tartrate Form A has an ¹H NMR spectrum comprising peaks substantially or essentially the same as shown in FIG. 25. The Tartrate Form A may be solvated, hemi-solvated or unsolvated.

In another embodiment, the present invention provides a pharmaceutical composition comprising the crystalline forms as described herein. The compositions described herein may be formulated for oral, subcutaneous, intravenous, or intramuscular administration. Preferably, the pharmaceutical composition is formulated for intravenous administration.

The psilocin compositions described herein may comprise a pharmaceutically effective amount of psilocin, in association with one or more pharmaceutically acceptable excipients including carriers, vehicles and diluents. The term “excipient” herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a solution for oral, parenteral, intradermal, subcutaneous, or topical application. Excipients can include, by way of illustration and not limitation, diluents, wetting agents, polymers, lubricants, stabilizers, and substances added to mask or counteract a disagreeable taste or odor, flavors, dyes, fragrances, and substances added to improve appearance of the composition. Acceptable excipients include (but are not limited to) stearic acid, magnesium stearate, sodium and calcium salts of phosphoric and sulfuric acids, magnesium carbonate, dextrin, mannitol, sorbitol, lactose, sucrose, starches, gelatin, polymers such as polyvinyl-pyrrolidone, polyvinyl alcohol, and polyethylene glycols, and other pharmaceutically acceptable materials. Examples of excipients and their use is described in Remington's Pharmaceutical Sciences, 20th Edition (Lippincott Williams & Wilkins, 2000). The choice of excipient will to a large extent depend on factors such as the mode of administration, the effect of the excipient on solubility and stability, and the nature of the dosage form.

In some embodiments the crystalline forms provided herein have greater aqueous solubility (solubility in water or saline) than psilocin base. For example, the solubility of the crystal forms may be at least about 0.25 mg/mL, 0.5 mg/mL, 1 mg/mL, 1.5 mg/mL, 2 mg/mL, 3 mg/mL, 4 mg/mL, 5 mg/mL, 6 mg/mL, 7 mg/mL, 8 mg/mL, 9 mg/mL, or at least about 10 mg/mL.

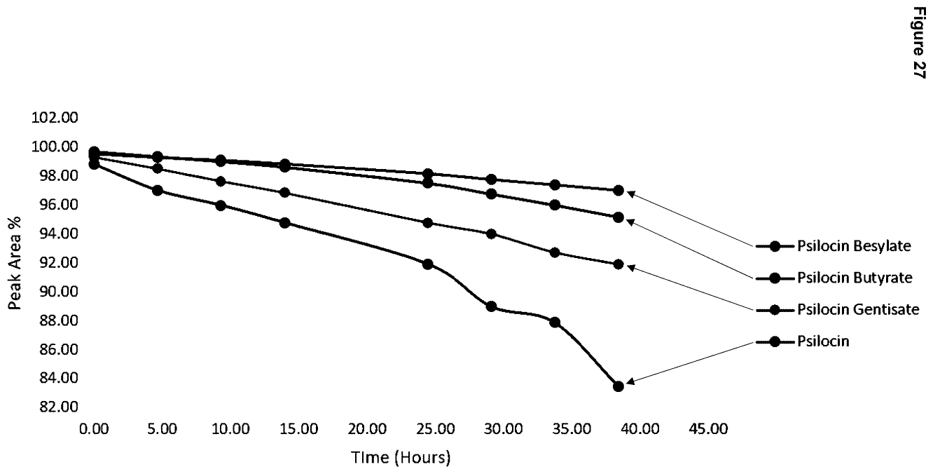

In some embodiments the crystalline forms provided herein have improved stability either in solid form or in solution (water or saline) compared to psilocin base in solution. For example, as demonstrated herein (see FIG. 27) the crystalline forms degrade at a much slower rate than psilocin in saline solution. In some embodiments when the crystalline forms are in an aqueous solution such as saline there is less than a 10% decrease in the amount of psilocin present over time (e.g. 36 hours) compared to at least a 15% decrease for psilocin base.

In other embodiments the crystalline forms described herein are stable for one day, one week, two weeks, one month, two months, three months, four months, five months, six months or at least one year during storage under long term stability condition of 300 C, 65% relative humidity as well as at accelerated/stress condition of 40-45° C. and 75% relative humidity.

In some embodiments the crystalline forms described herein are stable for at least two years under long term stability conditions of 300 C, 65% relative humidity as well as at accelerated/stress condition of 40-45° C. and 75% relative humidity.

While the crystalline forms described herein have improved stability compared to psilocin base it is contemplated that the stability can be further improved by formulating the crystalline forms with one or more excipients to reduce the effects of oxidation on the crystalline form, for example ascorbate, pyruvate, ascrorbyl palmitate, butylated hydroxytoluene, calcium stearate, citrate, potassium metabisulfite, propyl gallate, sodium metabisulfite, sodium thiosulfate, vitamin E, and sodium edetate.

In another embodiment, the present invention provides a method of producing a stable crystalline form and/or a crystalline from with improved solubility, comprising the steps of:
a) reacting psilocin with a pharmaceutically acceptable acid, as described herein, in a solvent; and
b) drying the resultant product of step a).

In some embodiments, the ratio of psilocin to acid is 1:5, 1:4, 1:3, 1:2, 1:1, 2:1, 3:1, 4:1, or 5:1 mol/mol. In some embodiments, the solvent may be selected from ethyl acetate or acetone. In some embodiments, the reaction between psilocin with the pharmaceutically acceptable acid is conducted at a lowered temperature, preferably from about 2-8° C. In some embodiments, the drying is conducted under vacuum at ambient temperature.

In another embodiment, the present invention provides a crystalline form or pharmaceutical composition, as described herein, useful for treating diseases and conditions such as psychological conditions, post-traumatic stress, attention deficit hyperactivity disorder, anxiety, addiction, depression, compulsion, IBS (irritable bowel syndrome), fibromyalgia, CRPS (complex regional pain syndrome), phantom limb, eating disorders, diabetes for example, diabetes associated with obesity and type 2 diabetes, neurological injuries, pain, for example nociplastic pain, and inflammatory conditions.

In some embodiments, the present invention provides a method of treating or preventing a disease or condition in a subject comprising administering to the subject the crystalline form or the pharmaceutical composition, as described herein.

In some embodiments, the present invention provides use of the crystalline form or pharmaceutical composition, as described herein, in the manufacture of a medicament for treating or preventing a disease or condition.

In some embodiments, the present invention provides the crystalline form or the pharmaceutical composition, as described herein, for use in treating or preventing a disease or condition in a subject.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In order that the present technology may be more clearly understood, preferred embodiments will be described with reference to the following examples

### EXAMPLES

The present invention will now be illustrated by the following examples, which are not to be construed as limiting the present invention in any manner and are only examples of the various embodiments described herein.

### Example 1: Salt and Cocrystal Screens

### Analysis

Materials exhibiting unique crystalline XRPD patterns are assigned sequential alphabetical characters as the default designation, if no other character types already pertain to the compound. Each uniquely identified material is assigned a new designation, which includes the chemical name of the guest used. The designation is tentatively associated with the term “Material” until the phase purity and chemical composition is determined through further characterization. Presence of psilocin, composition determination, and verification of phase uniformity are necessary before the term “Form” is used.

Attempts to index XRPD patterns were performed in this screen. Indexing and structure refinement are computational studies. Successful indexing of the pattern indicates that the sample is composed primarily or exclusively of a single crystalline phase.

### Characterization of Psilocin

Psilocin packaged in 36 pre-weighed vials was received as the starting materials. The initial characterization of the material is described below. The screening activities and characterization for the generated materials are discussed below.

One of the samples (pre-weighed vials) was used for XRPD, XRPD indexing, and ¹H NMR analyses.

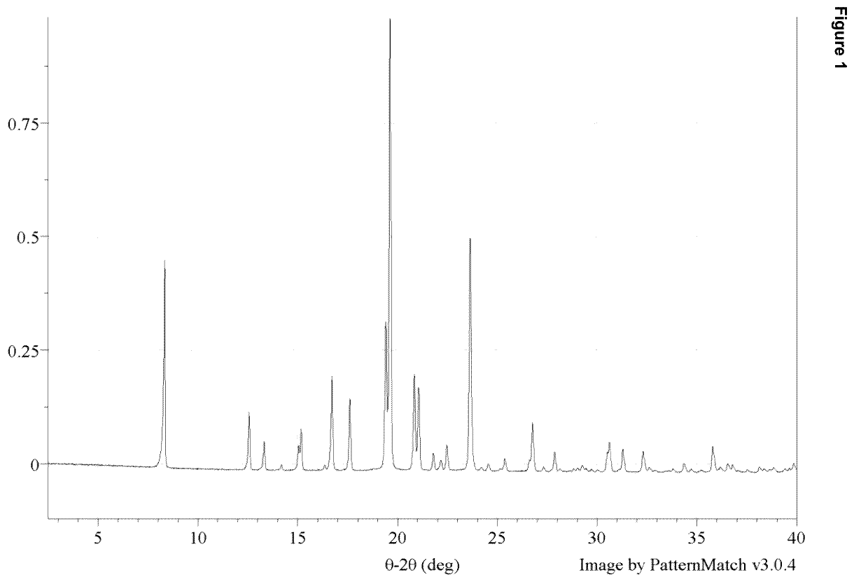

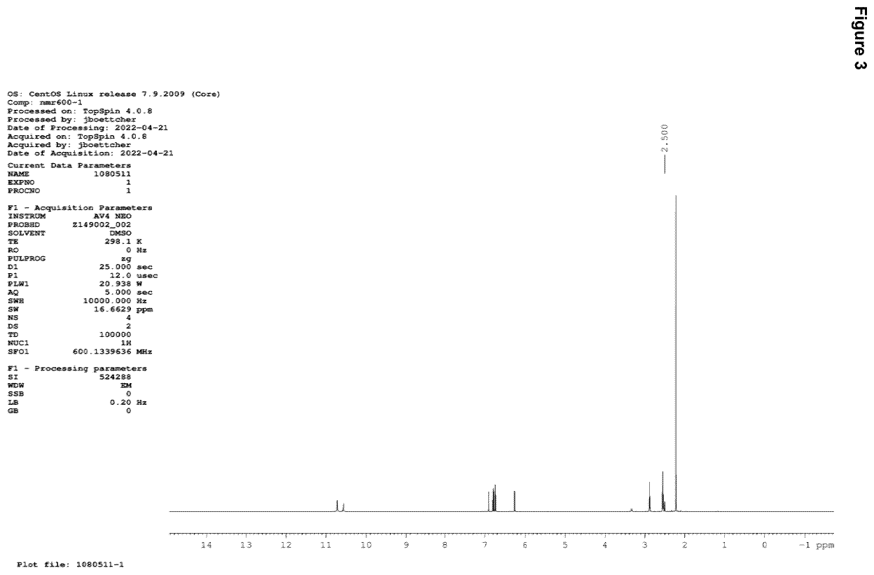

By XRPD, the psilocin is a crystalline material, as shown in FIG. 1. The XRPD pattern was successfully indexed, and the indexing solution is consistent with an unsolvated psilocin, as shown in FIG. 3.

The ¹H NMR spectrum of the psilocin is consistent with the chemical structure of psilocin, as shown in FIG. 4.

The approximate solubilities of psilocin were assessed in multiple solvents, as shown in Table 1 below.

TABLE 1
Approximate psilocin solubility

| | | Observation after |
|---|---|---|
| Solvent | Solubility (mg/mL)(a) | 1 day ambient storage |
| Acetone | 20 | No discoloration |
| ACN | 9 | No discoloration |
| Dioxane | 18 | No discoloration |
| EtOAc | 8 | No discoloration |
| EtOH | <1 | No discoloration |
| H₂0 | <1 | Discoloration (black suspension) |
| MeOH | <1 | No discoloration |
| THF | 18 | No discoloration |

(a)Solubilities are estimated at ambient temperature; if complete dissolution was not achieved, the value is reported as “<”. The actual solubility may be larger than the value calculated because of the use of solvent aliquots that were too large or due to a slow rate of dissolution.

Psilocin free base is poorly soluble in water and alcohols, but is relatively soluble in other organic solvents (≥8 mg/mL). The solubility estimates were utilized to design experiments for the salt screen. After ambient storage for 1 day, the sample in water showed a strong discoloration (black), while the other samples remained colorless.

### Salt/Cocrystal Screen of Psilocin

20 acids suitable for pharmaceutical salt development were selected, based on e.g., the calculated pKa (9.38, by ACD/pKa DB v11.01) of psilocin, and the solubilities of psilocin (see Table 1) and acids. 4 coformers were included for screening. All acids and coformers used in this study are summarized below.

TABLE 2
Summary of salt/cocrystal screen of psilocin

| | Acid/coformer | pKₐ₁ | GRAS |
|---|---|---|---|
| Acid | Acetic acid | 4.756 | Yes |
| | Aconitic acid | 2.80 | Yes |
| | Ascorbic acid | 4.17 | Yes |
| | Benzesulfonic acid | 0.17 | — |
| | Benzoic acid | 4.19 | Yes |
| | Butyric acid | 4.82 | — |
| | Citric acid | 3.128 | Yes |
| | Erythorbic acid | 2.1 | Yes |
| | Fumaric acid | 3.03 | — |
| | Gentisic acid | 2.93 | — |
| | Glutamic acid | 2.19 | Yes |
| | Glycolic acid | 3.83 | — |
| | Hydrochloric acid | −6 | Yes |
| | Maleic acid | 1.92 | — |
| | Phosphoric acid | 1.96 | Yes |
| | Pyrogluatmic acid | 3.32 | — |
| | Sorbic acid | 4.75 | Yes |
| | Succinic acid | 4.207 | Yes |
| | Sulfuric acid | −3 | Yes |
| | Tartaric acid | 3.02 | Yes |
| Coformer | Arginine | — | — |
| | Lysine | — | — |
| | Methyl paraben | — | Yes |
| | Nicotinamide | — | Yes |

For salt/cocrystal formation experiments, solvent-based techniques were employed, including evaporation, solvent-antisolvent addition, and slurry/trituration. Due to the stability issues of psilocin, most of experiments were performed at sub-ambient conditions (2-8° C.) or under N₂ flow. Materials isolated from the experiments were observed under PLM and analyzed by XRPD, if birefringence was observed. Most samples were dried under vacuum at ambient temperatures before XRPD analyses. The XRPD patterns were compared to the pattern of psilocin and reference patterns of the acids-coformers. Detailed experimental conditions, observations and results of the screening experiments are summarized in Table 3 and Table 4.

TABLE 3
Summary of salt screen of psilocin

| Solvent (RR)(a) | | | |
|---|---|---|---|
| (RR = reaction | | | XRPD |
| ratio) | Condition (b) | Observation | Result |
| acetic acid | 1. acid added to psilocin suspension in | 1. thin pink | — |
| (1:1) | EtOH, stirred at RT, 1 d | suspension | |
| | 2. FE (fast evaporation) under N2 | 2. gel | |
| | 3. added ACN, stirred at 2-8° C., 8 d | 3. brown gel w/solution | |
| | 1. acid added to psilocin suspension in | 1. white suspension, | Acetate |
| | EtOAc, stirred at 2-8° C., 2 d | B/E agg. | Form A |
| | 2. isolated solids dried under vacuum, | 2. — | |
| | RT, 1 d | | |
| | 1. acid added to psilocin suspension in | 1. thin suspension | — |
| | IPA, stirred at 2-8° C., 1 d | | |
| | 2. FE (fast evaporation) under N2 | 2. gel | |
| | 3. added iBuOAc, stirred at 2-8° C., 2 d | 3. gel w/solution | |
| aconitic acid | 1. acid solution in IPA added to | 1. white suspension | psilocin |
| (1:1) | psilocin, stirred at 2-8° C., 2 d | | |
| | 2. isolated solids dried under vacuum, | 2. — | |
| | RT, 6 h | | |
| ascorbic acid | 1. acid suspension in | 1. thin pink | — |
| (1:1) | MeOH added to psilocin, | suspension | |
| | stirred at 2-8° C., 1 d | | |
| | 2. FE under N2 | 2. gel | |
| | 3. added ACN, stirred at | 3. light yellow | |
| | 2-8° C., 4 d | suspension, no B/E | |
| | 4. stirred at 2-8° C., 7 d | 4. yellow | |
| | | suspension, no B/E | |
| | 1. acid suspension in EtOAc added to | 1. white suspension, B/E | psilocin + |
| | psilocin, stirred at 2-8° C., 1 d | particles | ascorbic |
| | 2. isolated solids dried under vacuum, | 2. — | acid |
| | RT, 1 d | | |
| benzenesulfonic | 1. acid solution in EtOAc added to | 1. white suspension | Besylate |
| acid | psilocin, stirred at 2-8° C., 1 d | | Material A |
| (1:1) | 2. isolated solids dried under vacuum, | 2. — | |
| | RT, 1 h | | |
| benzoic acid | 1. acid solution in IPA added to | 1. white suspension, | Benzoate |
| (1:1) | psilocin, stirred at 2-8° C., 2 d | tiny B/E particles | Form A |
| butyric acid | 1. added EtOAc and acid to psilocin, | 1. suspension, B/E | Butyrate |
| (1:1) | stirred at 2-8° C., 1 d | Agg | Material A |
| | 2. isolated solids dried under vacuum, | 2. — | |
| | RT, 1 d. | | |
| citric acid | 1. acid suspension in ACN added to | 1. white suspension, B/E | X-ray |
| (1:1) | psilocin, stirred at 2-8° C., 2 d | agg. (deliquescence | amorphous |
| | | observed on post- | |
| | | XRPD sample) | |
| | 1. acid solution in acetone added to | 1. white suspension, | psilocin |
| | psilocin, stirred at 2-8° C., 1 d | B/E agg. | |
| | 2. isolated solids dried under vacuum, | 2. — | |
| | RT, 1 d | | |
| erythorbic | 1. acid suspension in IPA added to | 1. suspension | erythorbic |
| acid (1:1) | psilocin, stirred at 2-8° C., 1 d | | acid |
| | 2. added MeOH, stirred at 2-8° C., 1 d | 2. thin suspension | |
| | 3. FE under N₂ purge, 1 h | 3. suspension | |
| | 4. isolated solids dried under vacuum, | 4. — | |
| | RT, 6 h | | |
| fumaric acid | 1. acid suspension in acetone added to | 1. white suspension, B/E | Fumarate |
| (1:1) | psilocin, stirred at 2-8° C., 2 d | agg. | Form A |
| | 1. acid suspension in IPA added to | 1. white suspension, | Fumarate |
| | psilocin, stirred at 2-8° C., 1 d | B/E particles | Form B |
| | 2. isolated solids dried under vacuum, | 2. — | |
| | RT, 1 d 1 | | |
| gentisic acid | 1. acid solution in EtOAc added to | 1. white suspension, | Gentisate |
| (2:1) | psilocin, stirred at 2-8° C., 2 d | B/E agg | Form A |
| | 1. acid solution in EtOAc added to | 1. white suspension | Gentisate |
| | psilocin, stirred at 2-8° C., 2 d | | Form A |
| | 2. isolated solids dried under vacuum, | 2. — | |
| | RT, 6 h | | |
| glutamic | 1. acid suspension in THF added to | 1. suspension, B/E agg. | glutamic |
| acid (1:1) | psilocin, stirred at 2-8° C., 1 d | | acid |
| | 2. isolated solids dried under vacuum, | 2. — | |
| | RT, 1 d | | |
| glycolic acid | 1. acid solution in EtOAc added to | 1. sticky solids w/ | psilocin |
| (1:1) | psilocin, stirred at 2-8° C., 2 d | suspension | |
| | 2. isolated solids dried under vacuum, | 2. — | |
| | RT, 6 h | | |
| HCl | 1. acid aqueous solution added to | 1. thin colorless | — |
| (1:2) | psilocin suspension in ACN, | suspension | |
| | stirred at 2-8° C., 1 d | | |
| | 2. FE under N₂ | 2. gel | |
| | 3. added EtOAc, stirred at 2-8° C., 4 d | 3. white suspension, no B/E | |
| | 4. stirred at 2-8° C., 7 d | 4. white suspension, no B/E | |
| HCl | 1. acid solution in EtOH added to | 1. pink suspension, | — |
| (1:1) | psilocin suspension in acetone, | no B/E | |
| | stirred at 2-8° C., 5 d | | |
| H₃PO₄ | 1. acid aqueous solution added to | 1. thin colorless suspension | — |
| (1:5) | psilocin suspension in ACN, stirred at | | |
| | 2-8° C., 1 d | | |
| | 2. FE under N₂ | 2. gel | |
| | 3. added MTBE, stirred at 2-8° C., 4 d | 3. gel w/solution, no B/E | |
| | 4. stirred at 2-8° C., 7 d | 4. sticky solids w/solution, | |
| | | no B/E | |
| H₃PO₄ | 1. acid solution in EtOAc added to | 1. white suspension, B/E & | X-ray |
| (1:1) | psilocin, stirred at 2-8° C., 5 d | no B/E particles | amorphous |
| | 2. isolated solids dried under vacuum, | 2. — | |
| | RT, 6 h | | |
| H₂SO₄ | 1. acid added to psilocin suspension in | 1. small amount of sticky | — |
| (1:1) | EtOAc, stirred at 2-8° C., 2 d | solids w/solution | |
| | 2. FE under N2 | 2. gel | |
| | 3. added MEK, stirred at 2-8° C., 2 d | 3. gel w/solution | |
| maleic acid | 1. acid suspension in EtOAc added to | 1. yellow suspension, B/E | X-ray |
| (1:2) | psilocin, stirred at 2-8° C., 2 d | agg., became gel when | amorphous + |
| | | kept at ambient shortly | minor |
| | 2. kept stirred at 2-8° C., 4 d | 2. yellow suspension | crystalline |
| | 3. decanted liquid, dried under vacuum | 3. sticky yellow | phase |
| | at RT for 4 h | solids, tiny B/E | |
| | | particles | |
| maleic acid | 1. acid suspension in acetone added to | 1. very thin light | — |
| (1:1) | psilocin, stirred at 2-8° C. for 1 d | yellow suspension | |
| | 2. added hexane | 2. precipitates | |
| | 3. stirred at 2-8° C., 1 d | 3. gel w/solution | |
| | 4. FE under N₂ | 4. gel | |
| | 5. added MTBE, stirred at 2-8° C., 3 d | 5. gel w/solution | |
| | 1. acid solution in ACN added to | 1. thin suspension | — |
| | psilocin, stirred at 2-8° C., 1 d | | |
| | 2. FE under N₂ | 2. gel | |
| | 3. added iPrOAc, stirred at 2-8° C., 3 d | 3. gel w/solution | |
| pyroglutamic | 1. acid suspension in IPA added to | 1. in progress | |
| acid (1:1) | psilocin, stirred at 2-8° C. | | |
| sorbic acid | 1. acid solution in acetone added to | 1. in progress | |
| (1:1) | psilocin, stirred at 2-8° C. | | |
| succinic acid | 1. acid suspension in IPA added to | 1. solution w/small amount | — |
| (1:1) | psilocin, stirred at 2-8° C., 2 d | of sticky solids | |
| | 1. acid suspension in EtOAc added to | 1. white suspension, B/E | Succinate |
| | psilocin, stirred at 2-8° C., 1 d | agg. | Material A + |
| | 2. isolated solids dried under vacuum, | 2. — | succinic |
| | RT, 1 d | | acid + |
| | | | psilocin |
| | | | (sample |
| | | | 8917-17- |
| | | | 04) |
| — | 1. sample 8917-17-04 and the post- | 1. white suspension | Succinate |
| | XRPD sample combined, added | | Material A + |
| | EtOAc, stirred at 2-8° C., 3 d | | 2nd |
| | 2. isolated solids dried under vacuum, | 2. — | phase(s) |
| | RT, 6 h | | |
| tartaric acid | 1. acid suspension in acetone added to | 1. white suspension, B/E | Tartrate |
| (1:1) | psilocin, stirred at 2-8° C., 1 d | agg. | Form A |
| | 2. isolated solids dried under vacuum, | 2. — | |
| | RT, 1 d | | |

(a)Reaction ratios (psilocin:acid, mol/mol) are approximate, with small excess of acid used if not specified.
(b): Times and temperatures are approximate.

TABLE 4
Summary of cocrystal screen of psilocin

| | | | XRPD |
|---|---|---|---|
| Solvent (RR)(a) | Condition (b) | Observation | Result |
| arginine | 1. coformer suspension in EtOH added | 1. suspension, B/E | arginine |
| (1:1) | to psilocin, stirred at 2-8° C., 1 d | agg. | |
| | 2. isolated solids dried under vacuum, | 2. — | |
| | RT, 1 d | | |
| lysine (1:1) | 1. coformer suspension in EtOH added | 1. thin suspension | psilocin + |
| | to psilocin, stirred at 2-8° C., 2 d | | lysine + |
| | 2. FE under N₂ purge, 1 h | 2. suspension | lysine |
| | 3. isolated solids dried under vacuum, | 3. — | hemihydrate |
| | RT, 6 h | | |
| methyl | 1. coformer solution in acetone added | 1. clear | psilocin |
| paraben | to psilocin, stirred at 2-8° C., 1 d | | |
| (1:1) | 2. FE under N₂ purge, 3 h | 2. suspension, B/E agg. | |
| | 3. isolated solids dried under vacuum, | 3. — | |
| | RT, 3 h | | |
| nicotinamide | 1. coformer solution in acetone added | 1. clear | psilocin + |
| (1:1) | to psilocin, stirred at 2-8° C., 1 d | | nicotinamide |
| | 2. FE under N₂ purge, 3 h | 2. suspension, B/E agg. | |
| | 3. isolated solids dried under vacuum, | 3. — | |
| | RT, 3 h | | |

(a)Reaction ratios (psilocin:coformer, mol/mol) are approximate, with small excess of coformer used if not specified.
(b): Times and temperatures are approximate.

Multiple crystalline materials with unique XRPD patterns were produced in the screen. These materials were further analyzed by ¹H NMR to confirm salt formation. The XRPD indexing and ¹H NMR results are summarized in Table 5.

TABLE 5
XRPD Indexing and NMR for Selected Materials

| Material | Analysis | Result |
|---|---|---|
| Acetate | XRPD | successfully indexed, consistent w/an unsolvated mono-acetate |
| Form | indexing | |
| A | ¹H NMR | generally consistent w/chemical structure of psilocin, containing 1.0 |
| | | mole of acetic acid and 0.03 moles of EtOAc, peak shift observed |
| | | compared to spectrum of psilocin |
| Benzoate | XRPD | successfully indexed, consistent w/an unsolvated mono-benzoate |
| Form | indexing | |
| A | ¹H NMR | generally consistent w/chemical structure of psilocin, containing 1.0 |
| | | mole of benzoic acid and 2.1 moles of IPA, peak shift observed |
| | | compared to spectrum of psilocin |
| Benzoate | ¹H NMR | generally consistent w/chemical structure of psilocin, containing 1.0 |
| Form | | mole of benzoic acid and 0.04 moles of IPA, peak shift observed |
| A, post-dried | | compared to spectrum of psilocin |
| Besylate | XRPD | successfully indexed, consistent w/an unsolvated mono-besylate |
| Material A | indexing | |
| | ¹H NMR | generally consistent w/chemical structure of psilocin, containing |
| | | benzenesulfonic acid, peak shift observed compared to spectrum of |
| | | psilocin |
| Butyrate | XRPD | successfully indexed, consistent w/an unsolvated mono-butyrate |
| Material A | indexing | |
| | ¹H NMR | generally consistent w/chemical structure of psilocin, containing butyric |
| | | acid, peak shift observed compared to spectrum of psilocin |
| amorphous | ¹H NMR | generally consistent w/chemical structure of psilocin, containing 1.5 |
| citrate | | moles of ACN and citric acid close to 1 mole (due to peaks overlapping, |
| | | only estimation is provided), peak shift observed compared to spectrum |
| | | of psilocin |
| Fumarate | XRPD | successfully indexed, may accommodate 1 mole of acetone for a hemi- |
| Form | indexing | fumarate |
| A | ¹H NMR | generally consistent w/chemical structure of psilocin, containing 0.5 |
| | | moles of fumaric acid and 0.7 moles of acetone, peak shift observed |
| | | compared to spectrum of psilocin |
| Fumarate | XRPD | successfully indexed, consistent w/an unsolvated hemi-fumarate |
| Form | indexing | |
| B | | |
| Fumarate | ¹H NMR | generally consistent w/chemical structure of psilocin, containing 0.5 |
| Form | | moles of fumaric acid and 0.2 moles of acetone, peak shift observed |
| B + 2nd | | compared to spectrum of psilocin |
| phase(s) | | |
| Gentisate | XRPD | successfully indexed, may accommodate 1 mole of water or half mole |
| Form | indexing | of EtOAc for a mono-gentisate |
| A | ¹H NMR | generally consistent w/chemical structure of psilocin, containing 1.0 |
| | | mole of gentisic acid and 1.0 mole of EtOAc, peak shift observed |
| | | compared to spectrum of psilocin |
| Gentisate | ¹H NMR | generally consistent w/chemical structure of psilocin, containing 1.0 |
| Form | | mole of gentisic acid and 0.4 mole of EtOAc, peak shift observed |
| A, post-dried | | compared to spectrum of psilocin |
| Tartrate | XRPD | successfully indexed, consistent w/an unsolvated mono-tartrate |
| Form | indexing | |
| A | ¹H NMR | generally consistent w/chemical structure of psilocin, containing 1.0 |
| | | mole of tartaric acid and 0.1 moles of acetone, peak shift observed |
| | | compared to spectrum of psilocin |

### Besylate Form A

Besylate Form A was generated from a salt formation experiment in EtOAc using 1:1 mol/mol psilocin and benzenesulfonic acid, followed by drying. The XRPD pattern (FIG. 4) was successfully indexed (FIG. 5). The indexing solution is consistent with an unsolvated mono-salt.

The ¹H NMR spectrum of Besylate Form A (FIG. 6) is generally consistent with that of psilocin, containing 1.0 mole of benzenesulfonic acid and 0.02 moles of EtOAc. Peak shifts are observed, compared with the spectrum of psilocin free base, indicative of salt formation.

The observed and prominent peak positions from the XPRD pattern of psilocin besylate Form A (FIG. 4) are provided in Tables 6 and 7.

TABLE 6
Observed XRPD peak positions for psilocin besylate Form A

| °2θ | d Space (Å) | Intensity (%) |
|---|---|---|
| 7.69 ± 0.20 | 11.487 ± 0.298 | 13 |
| 10.26 ± 0.20 | 8.615 ± 0.167 | 5 |
| 10.96 ± 0.20 | 8.066 ± 0.147 | 5 |
| 11.72 ± 0.20 | 7.545 ± 0.128 | 26 |
| 12.47 ± 0.20 | 7.093 ± 0.113 | 26 |
| 12.84 ± 0.20 | 6.889 ± 0.107 | 6 |
| 13.49 ± 0.20 | 6.558 ± 0.097 | 19 |
| 14.73 ± 0.20 | 6.009 ± 0.081 | 52 |
| 15.44 ± 0.20 | 5.734 ± 0.074 | 100 |
| 16.18 ± 0.20 | 5.474 ± 0.067 | 7 |
| 18.33 ± 0.20 | 4.836 ± 0.052 | 61 |
| 19.04 ± 0.20 | 4.657 ± 0.048 | 5 |
| 19.68 ± 0.20 | 4.507 ± 0.045 | 14 |
| 20.62 ± 0.20 | 4.304 ± 0.041 | 46 |
| 20.99 ± 0.20 | 4.229 ± 0.040 | 31 |
| 21.77 ± 0.20 | 4.079 ± 0.037 | 41 |
| 22.25 ± 0.20 | 3.992 ± 0.035 | 40 |
| 22.59 ± 0.20 | 3.933 ± 0.034 | 73 |
| 23.22 ± 0.20 | 3.828 ± 0.033 | 29 |
| 23.71 ± 0.20 | 3.750 ± 0.031 | 47 |
| 24.10 ± 0.20 | 3.690 ± 0.030 | 22 |
| 25.15 ± 0.20 | 3.538 ± 0.028 | 19 |
| 25.41 ± 0.20 | 3.502 ± 0.027 | 78 |
| 25.65 ± 0.20 | 3.470 ± 0.027 | 19 |
| 26.29 ± 0.20 | 3.387 ± 0.025 | 5 |
| 26.76 ± 0.20 | 3.329 ± 0.024 | 9 |
| 27.72 ± 0.20 | 3.216 ± 0.023 | 10 |
| 27.99 ± 0.20 | 3.185 ± 0.022 | 7 |
| 28.67 ± 0.20 | 3.111 ± 0.021 | 4 |
| 28.93 ± 0.20 | 3.084 ± 0.021 | 6 |
| 29.63 ± 0.20 | 3.012 ± 0.020 | 4 |
| 30.43 ± 0.20 | 2.935 ± 0.019 | 5 |
| 30.76 ± 0.20 | 2.904 ± 0.018 | 8 |
| 31.15 ± 0.20 | 2.869 ± 0.018 | 13 |
| 31.77 ± 0.20 | 2.814 ± 0.017 | 5 |
| 32.13 ± 0.20 | 2.783 ± 0.017 | 3 |
| 32.94 ± 0.20 | 2.717 ± 0.016 | 4 |
| 33.65 ± 0.20 | 2.661 ± 0.015 | 9 |
| 34.94 ± 0.20 | 2.566 ± 0.014 | 7 |
| 35.69 ± 0.20 | 2.514 ± 0.014 | 8 |
| 36.49 ± 0.20 | 2.460 ± 0.013 | 6 |

TABLE 7
Prominent XRPD peak positions for psilocin besylate Form A

| °2θ | d Space (Å) | Intensity (%) |
|---|---|---|
| 11.72 ± 0.20 | 7.545 ± 0.128 | 26 |
| 12.47 ± 0.20 | 7.093 ± 0.113 | 26 |
| 13.49 ± 0.20 | 6.558 ± 0.097 | 19 |
| 14.73 ± 0.20 | 6.009 ± 0.081 | 52 |
| 15.44 ± 0.20 | 5.734 ± 0.074 | 100 |
| 18.33 ± 0.20 | 4.836 ± 0.052 | 61 |
| 20.62 ± 0.20 | 4.304 ± 0.041 | 46 |
| 20.99 ± 0.20 | 4.229 ± 0.040 | 31 |
| 21.77 ± 0.20 | 4.079 ± 0.037 | 41 |
| 22.25 ± 0.20 | 3.992 ± 0.035 | 40 |
| 22.59 ± 0.20 | 3.933 ± 0.034 | 73 |
| 23.22 ± 0.20 | 3.828 ± 0.033 | 29 |
| 23.71 ± 0.20 | 3.750 ± 0.031 | 47 |
| 24.10 ± 0.20 | 3.690 ± 0.030 | 22 |
| 25.41 ± 0.20 | 3.502 ± 0.027 | 78 |

### Butyrate Form A

Butyrate Material A was generated from a salt formation experiment in EtOAc using 1:1 mol/mol psilocin and butyric acid, followed by drying. The XRPD pattern (FIG. 7) was successfully indexed (FIG. 8). The indexing solution suggests it can be an unsolvated mono-salt.

The ¹H NMR spectrum of Butyrate Form A (FIG. 9) is generally consistent with that of psilocin, containing 1.0 mole of butyric acid and <0.01 moles of EtOAc. Peak shifts are observed, compared with the spectrum of psilocin free base, indicative of salt formation.

The observed and prominent peak positions from the XPRD pattern of psilocin butyrate Form A (FIG. 7) are provided in Tables 8 and 9.

TABLE 8
Observed XRPD peak positions for psilocin butyrate Form A

| °2θ | d Space (Å) | Intensity (%) |
|---|---|---|
| 9.33 ± 0.20 | 9.471 ± 0.203 | 23 |
| 9.96 ± 0.20 | 8.874 ± 0.178 | 22 |
| 10.66 ± 0.20 | 8.292 ± 0.155 | 26 |
| 12.96 ± 0.20 | 6.825 ± 0.105 | 17 |
| 13.24 ± 0.20 | 6.682 ± 0.100 | 47 |
| 14.36 ± 0.20 | 6.163 ± 0.085 | 5 |
| 15.34 ± 0.20 | 5.771 ± 0.075 | 53 |
| 15.88 ± 0.20 | 5.576 ± 0.070 | 100 |
| 16.28 ± 0.20 | 5.440 ± 0.066 | 43 |
| 17.80 ± 0.20 | 4.979 ± 0.055 | 20 |
| 18.36 ± 0.20 | 4.828 ± 0.052 | 9 |
| 18.75 ± 0.20 | 4.729 ± 0.050 | 13 |
| 18.97 ± 0.20 | 4.674 ± 0.049 | 17 |
| 19.63 ± 0.20 | 4.519 ± 0.046 | 16 |
| 20.01 ± 0.20 | 4.434 ± 0.044 | 5 |
| 20.35 ± 0.20 | 4.360 ± 0.042 | |
| 20.95 ± 0.20 | 4.237 ± 0.040 | 38 |
| 21.44 ± 0.20 | 4.141 ± 0.038 | 7 |
| 21.98 ± 0.20 | 4.041 ± 0.036 | 22 |
| 22.32 ± 0.20 | 3.980 ± 0.035 | 20 |
| 22.80 ± 0.20 | 3.897 ± 0.034 | 5 |
| 23.31 ± 0.20 | 3.813 ± 0.032 | 20 |
| 23.77 ± 0.20 | 3.740 ± 0.031 | 7 |
| 24.41 ± 0.20 | 3.644 ± 0.029 | 8 |
| 24.61 ± 0.20 | 3.614 ± 0.029 | 20 |
| 25.46 ± 0.20 | 3.496 ± 0.027 | 7 |
| 25.60 ± 0.20 | 3.477 ± 0.027 | 9 |
| 26.22 ± 0.20 | 3.396 ± 0.025 | 7 |
| 26.67 ± 0.20 | 3.340 ± 0.025 | 16 |
| 27.79 ± 0.20 | 3.208 ± 0.023 | 38 |
| 29.07 ± 0.20 | 3.069 ± 0.021 | 4 |

TABLE 9
Prominent XRPD peak positions for psilocin butyrate Form A

| °2θ | d Space (Å) | Intensity (%) |
|---|---|---|
| 9.33 ± 0.20 | 9.471 ± 0.203 | 23 |
| 9.96 ± 0.20 | 8.874 ± 0.178 | 22 |
| 10.66 ± 0.20 | 8.292 ± 0.155 | 26 |
| 13.24 ± 0.20 | 6.682 ± 0.100 | 47 |
| 15.34 ± 0.20 | 5.771 ± 0.075 | 53 |
| 15.88 ± 0.20 | 5.576 ± 0.070 | 100 |
| 16.28 ± 0.20 | 5.440 ± 0.066 | 43 |
| 17.80 ± 0.20 | 4.979 ± 0.055 | 20 |
| 20.95 ± 0.20 | 4.237 ± 0.040 | 38 |
| 21.98 ± 0.20 | 4.041 ± 0.036 | 22 |
| 22.32 ± 0.20 | 3.980 ± 0.035 | 20 |
| 23.31 ± 0.20 | 3.813 ± 0.032 | 20 |
| 24.61 ± 0.20 | 3.614 ± 0.029 | 20 |
| 27.79 ± 0.20 | 3.208 ± 0.023 | 38 |

### Gentisate Form A

Gentisate Form A was generated from an experiment using 2:1 mol/mol psilocin and gensitic acid in EtOAc at 2-8° C. The XRPD pattern (FIG. 10) was successfully indexed (FIG. 11). Based on the indexing solution, Gentisate Form A may accommodate at least 1 mole of water or half mole of EtOAc, if it is a 1:1 salt.

The ¹H NMR spectrum of Gentisate Form A (FIG. 12) is generally consistent with that of psilocin, containing 1.0 mole of gentisic acid and 1.0 mole of EtOAc. Peak shifts are observed, compared with the spectrum of psilocin free base, indicative of salt formation.

Gentisate Form A was dried under vacuum at ambient temperature for 1 day and the post-dried sample maintains the same form.

The ¹H NMR spectrum of the post-dried Gentisate Form A (FIG. 12) is consistent with the spectrum before drying, while containing 0.4 moles of EtOAc. Additional materials of Gentisate Form A were generated for further analyses.

The observed and prominent peak positions from the XPRD pattern of gentisate Form A (FIG. 10) are provided in Tables 10 and 11.

TABLE 10
Observed XRPD peak positions for psilocin gentisate Form A

| °2θ | d Space (Å) | Intensity (%) |
|---|---|---|
| 7.74 ± 0.20 | 11.413 ± 0.294 | 16 |
| 9.01 ± 0.20 | 9.807 ± 0.217 | 11 |
| 11.01 ± 0.20 | 8.030 ± 0.145 | 10 |
| 12.29 ± 0.20 | 7.196 ± 0.117 | 15 |
| 12.77 ± 0.20 | 6.927 ± 0.108 | 35 |
| 13.15 ± 0.20 | 6.727 ± 0.102 | 12 |
| 13.80 ± 0.20 | 6.412 ± 0.092 | 17 |
| 14.08 ± 0.20 | 6.285 ± 0.089 | 33 |
| 15.52 ± 0.20 | 5.705 ± 0.073 | 82 |
| 15.80 ± 0.20 | 5.604 ± 0.070 | 100 |
| 15.98 ± 0.20 | 5.542 ± 0.069 | 31 |
| 16.11 ± 0.20 | 5.497 ± 0.068 | 22 |
| 16.51 ± 0.20 | 5.365 ± 0.065 | 83 |
| 17.30 ± 0.20 | 5.122 ± 0.059 | 45 |
| 18.07 ± 0.20 | 4.905 ± 0.054 | 19 |
| 18.58 ± 0.20 | 4.772 ± 0.051 | 58 |
| 19.13 ± 0.20 | 4.636 ± 0.048 | 17 |
| 19.39 ± 0.20 | 4.574 ± 0.047 | 26 |
| 19.56 ± 0.20 | 4.535 ± 0.046 | 25 |
| 20.95 ± 0.20 | 4.237 ± 0.040 | 55 |
| 21.64 ± 0.20 | 4.103 ± 0.037 | 32 |
| 22.18 ± 0.20 | 4.005 ± 0.036 | 15 |
| 22.45 ± 0.20 | 3.957 ± 0.035 | 15 |
| 23.03 ± 0.20 | 3.859 ± 0.033 | 17 |
| 23.38 ± 0.20 | 3.802 ± 0.032 | 53 |
| 23.98 ± 0.20 | 3.708 ± 0.030 | 91 |
| 24.74 ± 0.20 | 3.596 ± 0.029 | 63 |
| 24.95 ± 0.20 | 3.566 ± 0.028 | 24 |
| 25.19 ± 0.20 | 3.533 ± 0.028 | 44 |
| 25.71 ± 0.20 | 3.462 ± 0.026 | 15 |
| 26.08 ± 0.20 | 3.414 ± 0.026 | 16 |
| 26.47 ± 0.20 | 3.365 ± 0.025 | 20 |
| 27.28 ± 0.20 | 3.266 ± 0.023 | 13 |
| 27.81 ± 0.20 | 3.205 ± 0.023 | 31 |
| 28.41 ± 0.20 | 3.139 ± 0.022 | 55 |
| 28.80 ± 0.20 | 3.097 ± 0.021 | 41 |
| 30.13 ± 0.20 | 2.964 ± 0.019 | 11 |
| 30.66 ± 0.20 | 2.913 ± 0.019 | 8 |
| 31.90 ± 0.20 | 2.803 ± 0.017 | 8 |
| 32.16 ± 0.20 | 2.781 ± 0.017 | 9 |
| 32.57 ± 0.20 | 2.747 ± 0.016 | 11 |
| 33.37 ± 0.20 | 2.683 ± 0.016 | 10 |
| 33.75 ± 0.20 | 2.653 ± 0.015 | 7 |
| 34.77 ± 0.20 | 2.578 ± 0.014 | 7 |
| 35.29 ± 0.20 | 2.541 ± 0.014 | 7 |
| 36.25 ± 0.20 | 2.476 ± 0.013 | 11 |
| 36.80 ± 0.20 | 2.440 ± 0.013 | 11 |

TABLE 11
Prominent XRPD peak positions for psilocin gentisate Form A

| °2θ | d Space (Å) | Intensity (%) |
|---|---|---|
| 12.77 ± 0.20 | 6.927 ± 0.108 | 35 |
| 14.08 ± 0.20 | 6.285 ± 0.089 | 33 |
| 15.52 ± 0.20 | 5.705 ± 0.073 | 82 |
| 15.80 ± 0.20 | 5.604 ± 0.070 | 100 |
| 15.98 ± 0.20 | 5.542 ± 0.069 | 31 |
| 16.51 ± 0.20 | 5.365 ± 0.065 | 83 |
| 17.30 ± 0.20 | 5.122 ± 0.059 | 45 |
| 18.58 ± 0.20 | 4.772 ± 0.051 | 58 |
| 20.95 ± 0.20 | 4.237 ± 0.040 | 55 |
| 21.64 ± 0.20 | 4.103 ± 0.037 | 32 |
| 23.38 ± 0.20 | 3.802 ± 0.032 | 53 |
| 23.98 ± 0.20 | 3.708 ± 0.030 | 91 |
| 24.74 ± 0.20 | 3.596 ± 0.029 | 63 |
| 25.19 ± 0.20 | 3.533 ± 0.028 | 44 |
| 27.81 ± 0.20 | 3.205 ± 0.023 | 31 |
| 28.41 ± 0.20 | 3.139 ± 0.022 | 55 |
| 28.80 ± 0.20 | 3.097 ± 0.021 | 41 |

### Use of Acids in FDA-Approved IV Drugs

Benzenesulfonic acid, butyric acid, and gentisic acid have all been involved in development of FDA-approved IV drugs. Therefore they are deemed as potentially safe salt formers for IV administration.

Tracrium®, Atracurium Besylate, is an intermediate-duration, nondepolarizing, skeletal muscle relaxant for IV administration (https://www.rxlist.com/tracrium-drug.htm). Cleviprex®, Clevidipine Butyrate, is an IV dihydropyridine calcium channel blocker (https://www.cleviprex.com). AZEDRA®, (iobenguane I 131) injection, for IV use, contains sodium gentisate as an excipient (https://www.accessdata.fda.gov/drugsatfda_docs/label/2018/209607s0001bl.pdf).

### Acetate Form A

Acetate Form A was generated from a reaction between psilocin and acetic acid (1:1 mol/mol) in EtOAc. The solids were dried under vacuum at ambient temperature before analysis. The XRPD pattern (FIG. 13) was successfully indexed (FIG. 14). Based on the indexing solution, Acetate Form A can be an unsolvated material. The ¹H NMR spectrum of Acetate Form A (FIG. 15) is consistent with psilocin chemical structure, containing 1 mole of acetic acid and 0.03 moles of EtOAc. Peak shifts were observed compared with the spectrum of psilocin free base, indicative of salt formation. Therefore Acetate Form A is likely an unsolvated mono-salt.

### Benzoate Form A

Through a salt experiment between psilocin and benzoic acid (1:1 mol/mol) at 2-8° C., a new crystalline material was obtained, designated as psilocin Benzoate Form A. The XRPD pattern (FIG. 16) was successfully indexed (FIG. 17), and the indexing solution is consistent with an unsolvated 1:1 benzoate. The ¹H NMR spectrum of Benzoate Form A (FIG. 18) is generally consistent with that of psilocin, containing 1.0 mole of benzoic acid and 2.1 moles of IPA. Peak shifts are observed compared with the spectrum of psilocin free base, indicative of salt formation. Benzoate Form A was dried under vacuum at ambient temperature for 1 day and the post-dried sample maintains the same form (see, Table 12 below).

TABLE 12
Drying Studies for Selected Materials

| Starting material | Condition (a) | XRPD Result |
|---|---|---|
| Benzoate Form A | vacuum, RT, 1 d | Benzoate Form A |
| Fumarate Form A | vacuum, RT, 1 d | Fumarate Form B + 2nd phase(s) |
| Gentisate Form A | vacuum, RT, 1 d | Gentisate Form A |

(a): Times are approximate

The ¹H NMR spectrum of the post-dried Benzoate Form A is consistent with the spectrum before drying, containing 0.04 moles of IPA. These results indicate that Benzoate Form A may be an unsolvated mono-benzoate. The solubility of Benzoate Form A in ACN is lower than 0.25 mg/mL (see, Table 6), quite lower than that of the psilocin free base in ACN (9 mg/mL—see, Table 2). The solubility of Benzoate Form A in DMSO is greater than 12 mg/mL (see, Table 13). Therefore DMSO may be considered as a solvent for HPLC analysis for stability study.

TABLE 13
Drying Studies for Selected Materials

| Solvent | Solubility (mg/mL) (a) |
|---|---|
| ACN | <0.25 |
| DMSO | >12 |

(a): Solubilities are estimated at ambient temperature; if complete dissolution was not achieved, the value is reported as “<”; if complete dissolution was achieved with one aliquot of solvent, the value is reported as “>”. The actual solubility may be larger than the value calculated because of the use of solvent aliquots that were too large or due to a slow rate of dissolution.

### Fumarate Form A

Fumarate Form A was generated from an experiment using 1:1 mol/mol psilocin and fumaric acid in acetone at 2-8° C. The XRPD pattern of Fumarate Form A (FIG. 19) was successfully indexed (FIG. 20). Based on the indexing solution, Fumarate Form A may accommodate 1 mole of acetone, if it is a hemi-fumarate. The ¹H NMR spectrum of Fumarate Form A (FIG. 22) is generally consistent with that of psilocin, containing 0.5 moles of fumaric acid and 0.7 moles of acetone. Peak shifts are observed, compared with the spectrum of psilocin free base, indicative of salt formation. Fumarate Form A was dried under vacuum at ambient temperature for 1 day, and the sample converted to a mixture of Fumarate Form B and minor unknown secondary phase(s). The ¹H NMR spectrum of this mixture is consistent with that of Fumarate Form A, containing 0.2 moles of acetone. From an additional salt experiment with fumaric acid in IPA, followed by drying under vacuum, a single phase of Fumarate Form B was generated. This XRPD pattern was successfully indexed and the indexing solution is consistent with an unsolvated hemi-fumarate.

### Tartrate Form A

Psilocin Tartrate Form A was generated from a reaction between psilocin and tartaric acid (1:1 mol/mol) in acetone. The solids were dried under vacuum at ambient temperature before analysis. The XRPD pattern (FIG. 23) was successfully indexed (FIG. 24). Based on the indexing solution, Tartrate Form A can be unsolvated for a 1:1 salt. The ¹H NMR spectrum of Tartrate Form A (FIG. 25) is consistent with psilocin chemical structure, containing 1 mole of tartaric acid and 0.1 moles of acetone. Peak shifts were observed compared with the spectrum of psilocin free base, indicative of salt formation. Therefore Tartrate Form A is likely an unsolvated mono-salt.

### Succinate Material A

A salt experiment using 1:1 mol/mol psilocin and succinic acid in EtOAc generated a mixture of a new material containing succinic acid and psilocin. This new material is designated as Succinate Material A. To complete the salt reaction and remove residual psilocin and acid, the mixture was re-slurried in EtOAc. In the re-slurried material, psilocin and succinic acid are not present. However an additional phase is observed. This mixture was not further analyzed.

Experiments with Other Acids/Coformers

New crystalline materials were not observed in experiments with aconitic acid, ascorbic acid, citric acid, erythorbic acid, glutamic acid, glycolic acid, hydrochloric acid, maleic acid, phosphoric acid, pyrogluamic acid, sorbic acid, sulfuric acid, arginine, lysine, methyl paraben, and nicotinamide (see, Tables 3 and 4).

From an experiment using 1:1 mol/mol psilocin and citric acid, a sample exhibiting birefringence was observed. However by XRPD, the sample appears to be X-ray amorphous. By visual observation, the post-XRPD sample deliquesced, suggesting the sample may be physically unstable at ambient temperature. The ¹H NMR spectrum of the sample is generally consistent with that of psilocin, containing 1.5 moles of ACN and citric acid close to 1 mole (estimated result due to peaks overlapping). Peak shifts are observed, compared with the spectrum of psilocin free base, indicative of salt formation.

From an experiment using 1:2 mol/mol psilocin and maleic acid at 2-8° C., a yellow suspension showing birefringence was observed. However the solids became gel shortly after the sample was taken to ambient temperature. The sample was kept at 2-8° C. for further stirring and a yellow suspension was obtained again. The solids were immediately isolated at ambient temperature followed by drying under vacuum at ambient temperature. By XRPD, the final sample appears to be a mixture of amorphous material with a minor crystalline phase, which is not consistent with maleic acid or psilocin. This mixture was not further studied.

### Example 2. Methods

### Approximate Solubility

Weighed samples were treated with aliquots of designated solvent at ambient temperature. Complete dissolution of the test material was determined by visual inspection. Solubility was estimated based on the total solvent volume used to provide the complete dissolution. If complete dissolution was achieved by only one aliquot addition, the value is reported as “larger than”; if complete dissolution was not achieved, the value is reported as “less than”. The actual solubility may be greater than the value calculated because of the use of solvent aliquots that were too large or due to a slow rate of dissolution.

### Fast Evaporation (FE)

Solutions were prepared in selected solvents, and allowed to evaporate at ambient temperature from uncapped vials.

### X-ray Powder Diffraction (XRPD)

XRPD patterns were collected with a PANalytical X'Pert PRO MPD or Empyrean diffractometer using an incident beam of Cu radiation produced using an Optix long, fine-focus source. An elliptically graded multilayer mirror was used to focus Cu Kα X-ray radiation through the specimen and onto the detector. Prior to the analysis, a silicon specimen (NIST SRM 640f) was analyzed to verify the observed position of the Si (111) peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5.

The XRPD data presented herein include x-ray diffraction patterns with labeled peaks and tables with peak lists. The range of data collected is typically provided in the scientific report in which the data were initially reported, and is instrument dependent. Under most circumstances, peaks within the range of up to about 30° 2θ were selected. Rounding algorithms were used to round each peak to the nearest 0.1° or 0.01° 2θ, depending upon the instrument used to collect the data and/or the inherent peak resolution. The location of the peaks along the x-axis (° 2θ) in both the figures and the tables were rounded to one or two significant figures after the decimal point based upon the above criteria. Peak position variabilities are given to within ±0.2° 2θ based upon recommendations outlined in the USP discussion of variability in x-ray powder diffraction (USP-NF 2022, Issue 2, <941>, Characterization of Crystalline and Partially Crystalline Solids by X-Ray Powder Diffraction (XRPD), GUID-14EBB55E-0D24-45A1-A84FFE4DCAAEE3E8_2_en-US, official 1 May 2022.]. The accuracy and precision associated with any particular measurement reported herein has not been determined. Moreover, third party measurements on independently prepared samples on different instruments may lead to variability which is greater than ±0.2° 2θ. For d-space listings, the wavelength used to calculate d-spacings was 1.5405929 Å, the Cu-Kα1 wavelength (Phys. Rev. A56(6) 4554-4568 (1997)). Variability associated with d-spacing estimates was calculated from the USP recommendation, at each d-spacing, and provided in the respective data tables.

Per USP guidelines, variable hydrates and solvates may display peak variances greater than 0.2° 2θ and therefore peak variances of 0.2° 2θ are not applicable to these materials.

For samples with only one XRPD pattern and no other means to evaluate whether the sample provides a good approximation of the powder average, peak tables contain data identified only as “Prominent Peaks”. These peaks are a subset of the entire observed peak list. Prominent peaks are selected from observed peaks by identifying preferably non-overlapping, low-angle peaks, with strong intensity.

If multiple diffraction patterns are available, then assessments of particle statistics (PS) and/or preferred orientation (PO) are possible. Reproducibility among XRPD patterns from multiple samples analyzed on a single diffractometer indicates that the particle statistics are adequate. Consistency of relative intensity among XRPD patterns from multiple diffractometers indicates good orientation statistics. Alternatively, the observed XRPD pattern may be compared with a calculated XRPD pattern based upon a single crystal structure, if available. Two dimensional scattering patterns using area detectors can also be used to evaluate PS/PO. If the effects of both PS and PO are determined to be negligible, then the XRPD pattern is representative of the powder average intensity for the sample and prominent peaks may be identified as “Representative Peaks”.

### Proton Solution Nuclear Magnetic Resonance Spectroscopy (¹H NMR)

The proton solution NMR spectra were acquired with a Bruker AVANCE 600 MHz Spectrometer using DMSO-d6. The specific acquisition parameters are listed on the plot of the first full spectrum of the figures.

### Polarized Light Microscopy (PLM)

Light microscopy was performed using a Leica MZ12.5 stereomicroscope. Samples were observed using 0.8-10× objectives with crossed polarizers and a first order red compensator. Samples were either viewed in situ or in a drop of mineral oil.

### XRPD Indexing

The high-resolution XRPD patterns were indexed using X'Pert High Score Plus 2.2a (2.2.1) or TRIADS® in this study. Indexing and structure refinement are computational studies. Agreement between the allowed peak positions, marked with red bars, and the observed peaks indicates a consistent unit cell determination. Successful indexing of the pattern indicates that the sample is composed primarily of a single crystalline phase. Space groups consistent with the assigned extinction symbol, unit cell parameters, and derived quantities are tabulated below each figure showing tentative indexing solution. To confirm the tentative indexing solution, the molecular packing motifs within the crystallographic unit cells must be determined. No attempts at molecular packing were performed.

### Example 4: Comparison of Psilocin Salt and Psilocin Free Base Solubility in Saline

Psilocin besylate, psilocin butyrate, psilocin gentisate, and psilocin free base were prepared at 1.0 mg/mL in saline. Psilocin free base was also prepared at 0.1 mg/mL. Solubility of material in solution was observed and pH was recorded (Table 13)

TABLE 13
Solubility and pH

| Material | Appearance in solution | pH |
|---|---|---|
| Psilocin besylate 1.0 mg/mL | Visually soluble, clear solution | 5.010 |
| Psilocin butyrate 1.0 mg/mL | Visually soluble, clear solution | 6.281 |
| Psilocin Gentisate 1.0 mg/mL | Visually soluble, clear solution | 5.726 |
| Psilocin free base 1.0 mg/mL | Cloudy solution, not soluble | 9.395 |
| Psilocin free base 0.1 mg/mL | Visually soluble, clear solution | 9.053 |

It is apparent that the psilocin salts are substantially more soluble in saline than psilocin free base.

### Example 5: Psilocin Salt and Psilocin Free Base Stability in Saline

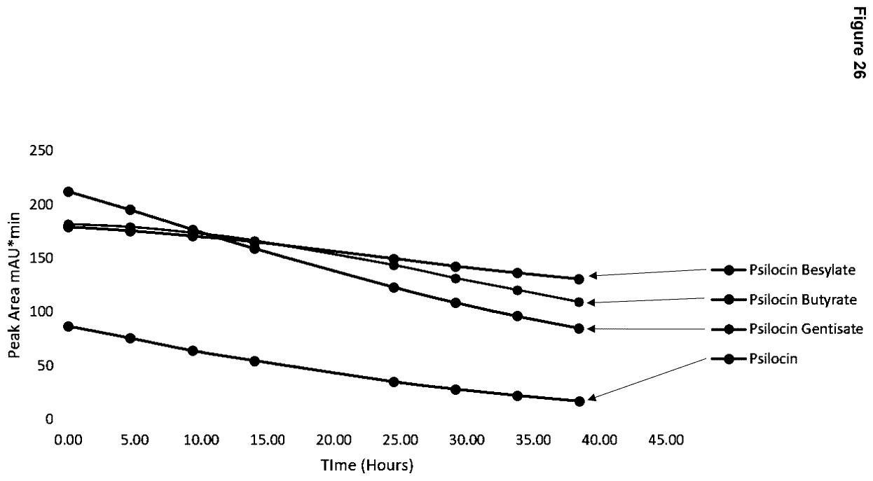

Psilocin besylate, psilocin butyrate, psilocin gentisate, and psilocin free base were prepared at 1.0 mg/mL in saline. Samples were filtered through a 0.2 μm PTFE filter. Solutions were analyzed at various time points over 38 hours using the HPLC conditions set out in the tables 14 and 15. The psilocin salts are substantially more stable over time than psilocin (see FIGS. 26 and 27).

TABLE 14
HPLC Conditions

| Column | Sielc Primesep 100, 4.6 × 150 mm, 3 μm | |
|---|---|---|
| Mobile Phase A | 90:10:0.3 H20/ACN/H2SO4 | |
| Mobile Phase B | 100% ACN | |
| Needle Wash | 100% CAN | |
| Column Temp | 50° | C. |
| Autosampler Temp | Ambient | |
| Detector wavelength | 219 | nm |
| Flow rate | 1.5 | mL/min |
| Injection Volume | 3 | μL |
| Run Time | 45 | min |

| | |
|---|---|
| Elution Mode | Gradient |

TABLE 15
HPLC gradient

| Time (min) | Mobile Phase A % | Mobile Phase B % |
|---|---|---|
| 0 | 100 | 0 |
| 7 | 100 | 0 |
| 13 | 85 | 15 |
| 28 | 75 | 25 |
| 35 | 75 | 25 |
| 35.1 | 100 | 0 |
| 45 | 100 | 0 |

### Example 6: Prophetic Example—Chemical Stability

Solid-state stability may be assessed using a temperature/humidity control chamber. A sample of each crystalline form is placed in the chamber and exposed to various temperatures and humidities, for example 25° C./60% RH, 40° C./75% RH, 70° C./75% RH, and/or irradiated with a Xenon lamp. The crystalline form, thermal behavior, purity and/or weight change of the resultant sample after the exposure or irradiation may be evaluated by using one or more of XRPD, thermogravity/differential thermal analysis, differential scanning calorimetry, high performance liquid chromatography, or a microbalance.

It is anticipated that each crystalline form will be stable. For example, in the solid-state stability study after storage at 25° C./60% RH, or 40° C./75% RH, or 70° C./75% RH for one week, two weeks, one month, or two months the crystalline forms described herein will be chemically and physically stable In addition, it is anticipated that fewer degradation products are found in the crystalline forms compared to psilocin. In this context purity can be determined by HPLC measurement and it is anticipated that degradation products will be less than 2%, 5%, 10% or 15% of the total crystalline form after storage at 25° C./60% RH, or 40° C./75% RH, or 70° C./75% RH for one week, two weeks, one month, or two months.

### Example 7: Prophetic Example—Photostability

Photostability experiments will be performed on approximately 3 mm depth of the solid psilocin crystalline forms and a solution of 0.2 mg/mL of the free base in water. Before dissolution the water will, be purged with nitrogen for 30 minutes to prevent oxidative degradation. Duplicate vials will be prepared for each sample, where one is exposed to light and the other to act as a control, which is wrapped in foil for the duration of the experiment. The sample will exposed at an iridescence level of, for example 500 W/m2 (300-800 nm) for the equivalent of 1 week of bright sunlight. Observations will be made before and after the exposure for the free base psilocin salt, and each crystalline form. The purity analysis will performed post exposure for all samples at 0.2 mg/mL of the free base using HPLC. The X-ray powder diffraction will be performed on the solid psilocin salt samples before and after exposure.

Similar experiments may be performed to compare photostability levels in clear glass to amber glass vials and to account for the presence or absence of nitrogen.

It is anticipated that the purity and stability of the solid samples after light exposure will not change when compared to pre-exposure. It is also anticipated that the XRPD analysis will also find that the samples will not change crystal form after the photostability experiments.

It is expected that the crystalline forms will all show a greater stability in the presence of light in comparison to free base psilocin. It is known that the purity of a free base in solution post exposure was decreases substantially (drops to around 35%), in contrast it is expected that the crystalline forms will retain a purity >75%, or >90% by HPLC after light exposure.

### Example 8: Prophetic Example—Forced Degradation

A test will be carried out to assess the stability of the psilocin crystalline forms and free base psilocin to oxidative degradation. Forced degradation of the psilocin salts will be performed in H₂O₂, for example 0.3% H₂O₂ to test the oxidative stability of each crystalline form. The appropriate volume of H₂O₂ will be added to a pre-weighed sample of the crystalline form in an amber vial (or other vial shieled from light) to give a maximum concentration of, for example 0.2 mg/mL of psilocin (free base equivalent). The samples will be stored at 25° C. and the purity of each sample was assessed periodically thereafter by HPLC. For example the samples may be assessed at 0, 1, 6, and 24 hours using HPLC.

It is expected that in H₂O₂ the rate of degradation will be slower for the crystalline forms compared to free base psilocin, this will demonstrate that the crystalline forms will have a superior shelf-life stability, and resistance to oxidative degradation.

1. A crystalline form of a pharmaceutically acceptable salt of psilocin (4-hydroxy-N,N-dimethyltryptamine), or a cocrystal of psilocin (4-hydroxy-N,N-dimethyltryptamine) and a coformer.

2. The crystalline form of claim 1, wherein the pharmaceutically acceptable salt is an acid.

3. The crystalline form of claim 2, wherein the acid or coformer is selected from one or more of acetic acid, aconitic acid, ascorbic acid, benzenesulfonic acid, benzoic acid, butyric acid, citric acid, erythorbic acid, fumaric acid, gentisic acid, glutamic acid, glycolic acid, hydrochloric acid, maleic acid, phosphoric acid, pyrogluamic acid, sorbic acid, succinic acid, sulfuric acid, tartaric acid, arginine, lysine, methyl paraben, nicotinamide and ethyl acetate.

4. The crystalline form of claim 3, wherein the acid is benzenesulfonic acid.

5. The crystalline form of claim 4, wherein the crystalline form is Besylate Form A and exhibits XRPD (X-ray power diffraction) peaks at about 15.44° 2θ±0.20, 18.33° 2θ±0.20, and 25.41° 2θ±0.20.

6. The crystalline form of claim 4, wherein the crystalline form is Besylate Form A and exhibits XRPD (X-ray power diffraction) peaks at about 14.73° 2θ±0.20, 15.44° 2θ±0.20, 18.33° 2θ±0.20, 22.59° 2θ±0.20, and 25.41° 2θ±0.20.

7. The crystalline form of claim 4, wherein the crystalline form is Besylate Form A and exhibits XRPD (X-ray power diffraction) peaks at about 11.72° 2θ±0.20, 12.47° 2θ±0.20, 13.49° 2θ±0.20, 14.73° 2θ±0.20, 15.44° 2θ±0.20, 18.33° 2θ±0.20, 20.62° 2θ±0.20, 20.99° 2θ±0.20, 21.77° 2θ±0.20, 22.25° 2θ±0.20, 22.59° 2θ±0.20, 23.22° 2θ±0.20, 23.71° 2θ±0.20, 24.10° 2θ±0.20, and 25.41° 2θ±0.20.

8. The crystalline form of claim 4, wherein the crystalline form is Besylate Form A and exhibits XRPD (X-ray power diffraction) peaks at about 7.69° 2θ±0.20, 10.26° 2θ±0.20, 10.96° 2θ±0.20, 11.72° 2θ±0.20, 12.47° 2θ±0.20, 12.84° 2θ±0.20, 13.49° 2θ±0.20, 14.73° 2θ±0.20, 15.44° 2θ±0.20, 16.18° 2θ±0.20, 18.33° 2θ±0.20, 19.04° 2θ±0.20, 19.68° 2θ±0.20, 20.62° 2θ±0.20, 20.99° 2θ±0.20, 21.77° 2θ±0.20, 22.25° 2θ±0.20, 22.59° 2θ±0.20, 23.22° 2θ±0.20, 23.71° 2θ±0.20, 24.10° 2θ±0.20, 25.15° 2θ±0.20, 25.41° 2θ±0.20, 25.65° 2θ±0.20, 26.29° 2θ±0.20, 26.76° 2θ±0.20, 27.72° 2θ±0.20, 27.99° 2θ±0.20, 28.67° 2θ±0.20, 28.93° 2θ±0.20, 29.63° 2θ±0.20, 30.43° 2θ±0.20, 30.76° 2θ±0.20, 31.15° 2θ±0.20, 31.77° 2θ±0.20, 32.13° 2θ±0.20, 32.94° 2θ±0.20, 33.65° 2θ±0.20, 34.94° 2θ±0.20, 35.69° 2θ±0.20, and 36.49° 2θ±0.20.

9. The crystalline form of any one of claims 4 to 8, wherein the crystalline form is Besylate Form A characterized by an X-ray powder diffraction spectrum substantially as depicted in FIG. 4.

10. The crystalline form of claim 3, wherein the acid is butyric acid.

11. The crystalline form of claim 10, wherein the crystalline form is Butyrate Form A and exhibits XRPD (X-ray power diffraction) peaks at about 13.24° 2θ±0.20, 15.34° 2θ±0.20, and 15.88° 2θ±0.20.

12. The crystalline form of claim 10, wherein the crystalline form is Butyrate Form A and exhibits XRPD (X-ray power diffraction) peaks at about 13.24° 2θ±0.20, 15.34° 2θ±0.20, 15.88° 2θ±0.20, 16.28° 2θ±0.20, 20.95° 2θ±0.20, and 27.79° 2θ±0.20.

13. The crystalline form of claim 10, wherein the crystalline form is Butyrate Form A and exhibits XRPD (X-ray power diffraction) peaks at about 9.33° 2θ±0.20, 9.96° 2θ±0.20, 10.66° 2θ±0.20, 13.24° 2θ±0.20, 15.34° 2θ±0.20, 15.88° 2θ±0.20, 16.28° 2θ±0.20, 17.80° 2θ±0.20, 20.95° 2θ±0.20, 21.98° 2θ±0.20, 22.32° 2θ±0.20, 23.31° 2θ±0.20, 24.61° 2θ±0.20, and 27.79° 2θ±0.20.

14. The crystalline form of claim 10, wherein the crystalline form is Butyrate Form A and exhibits XRPD (X-ray power diffraction) peaks at about 9.33° 2θ±0.20, 9.96° 2θ±0.20, 10.66° 2θ±0.20, 12.96° 2θ±0.20, 13.24° 2θ±0.20, 14.36° 2θ±0.20, 15.34° 2θ±0.20, 15.88° 2θ±0.20, 16.28° 2θ±0.20, 17.80° 2θ±0.20, 18.36° 2θ±0.20, 18.75° 2θ±0.20, 18.97° 2θ±0.20, 19.63° 2θ±0.20, 20.01° 2θ±0.20, 20.35° 2θ±0.20, 20.95° 2θ±0.20, 21.44° 2θ±0.20, 21.98° 2θ±0.20, 22.32° 2θ±0.20, 22.80° 2θ±0.20, 23.31° 2θ±0.20, 23.77° 2θ±0.20, 24.41° 2θ±0.20, 24.61° 2θ±0.20, 25.46° 2θ±0.20, 25.60° 2θ±0.20, 26.22° 2θ±0.20, 26.67° 2θ±0.20, 27.79° 2θ±0.20, and 29.07° 2θ±0.20.

15. The crystalline form of any one of claims 8 to 10, wherein the crystalline form is Butyrate Form A characterized by an X-ray powder diffraction spectrum substantially as depicted in FIG. 7.

16. The crystalline form of claim 3, wherein the acid is gentisic acid.

17. The crystalline form of claim 16, wherein the crystalline form is Gentisate Form A and exhibits XRPD (X-ray power diffraction) peaks at about 15.80° 2θ±0.20, 16.51° 2θ±0.20, and 23.98° 2θ±0.20.

18. The crystalline form of claim 16, wherein the crystalline form is Gentisate Form A and exhibits XRPD (X-ray power diffraction) peaks at about 15.52° 2θ±0.20, 15.80° 2θ±0.20, 16.51° 2θ±0.20, 23.98° 2θ±0.20, and 24.74° 2θ±0.20.

19. The crystalline form of claim 16, wherein the crystalline form is Gentisate Form A and exhibits XRPD (X-ray power diffraction) peaks at about 12.77° 2θ±0.20, 14.08° 2θ±0.20, 15.52° 2θ±0.20, 15.80° 2θ±0.20, 15.98±0.20, 16.51° 2θ±0.20, 17.30° 2θ±0.20, 18.58° 2θ±0.20, 20.95° 2θ±0.20, 21.64° 2θ±0.20, 23.38° 2θ±0.20, 23.98° 2θ±0.20, 24.74° 2θ±0.20, 25.19° 2θ±0.20, 27.81° 2θ±0.20, 28.41° 2θ±0.20, and 28.80° 2θ±0.20.

20. The crystalline form of claim 16, wherein the crystalline form is Gentisate Form A and exhibits XRPD (X-ray power diffraction) peaks at about 7.74° 2θ±0.20, 9.01° 2θ±0.20, 11.01° 2θ±0.20, 12.29° 2θ±0.20, 12.77° 2θ±0.20, 13.15° 2θ±0.20, 13.80° 2θ±0.20, 14.08° 2θ±0.20, 15.52° 2θ±0.20, 15.80° 2θ±0.20, 15.98° 2θ±0.20, 16.11° 2θ±0.20, 16.51° 2θ±0.20, 17.30° 2θ±0.20, 18.07° 2θ±0.20, 18.58° 2θ±0.20, 19.13° 2θ±0.20, 19.39° 2θ±0.20, 19.56° 2θ±0.20, 20.95° 2θ±0.20, 21.64° 2θ±0.20, 22.18° 2θ±0.20, 22.45° 2θ±0.20, 23.03° 2θ±0.20, 23.38° 2θ±0.20, 23.98° 2θ±0.20, 24.74° 2θ±0.20, 24.95° 2θ±0.20, 25.19° 2θ±0.20, 25.71° 2θ±0.20, 26.08° 2θ±0.20, 26.47° 2θ±0.20, 27.28° 2θ±0.20, 27.81° 2θ±0.20, 28.41° 2θ±0.20, 28.80° 2θ±0.20, 30.13° 2θ±0.20, 30.66° 2θ±0.20, 31.90° 2θ±0.20, 32.16° 2θ±0.20, 32.57° 2θ±0.20, 33.37° 2θ±0.20, 33.75° 2θ±0.20, 34.77° 2θ±0.20, 35.29° 2θ±0.20, 36.25° 2θ±0.20, and 36.80° 2θ±0.20.

21. The crystalline form of any one of claims 16 to 20, wherein the crystalline form is Gentisate Form A characterized by an X-ray powder diffraction spectrum substantially as depicted in FIG. 10.

22. The crystalline form of claim 3, wherein the acid is benzoic acid.

23. The crystalline form of any one of claims 16 to 18, wherein the crystalline form is Benzoate Form A characterized by an X-ray powder diffraction spectrum substantially as depicted in FIG. 16.

24. The crystalline form of claim 3, wherein the acid is fumaric acid.

25. The crystalline form of any one of claims 20 to 22, wherein the crystalline form is Fumarate Form A characterized by an X-ray powder diffraction spectrum substantially as depicted in FIG. 19.

26. The crystalline form of claim 3, wherein the acid is tartaric acid.

27. The crystalline form of any one of claims 20 to 22, wherein the crystalline form is Tartrate Form A characterized by an X-ray powder diffraction spectrum substantially as depicted in FIG. 23.

28. The crystalline from of any one of claims 1 to 27 wherein the crystalline form is stable after storage at 25° C., 40° C., or 70° C. for one day, one week, two weeks, one month, two months, three months, four months, five months, 6 months or at least one year.

29. The crystalline from of any one of claims 1 to 28 wherein the crystalline form is more stable in water or saline compared to psilocin base in water or saline.

30. The crystalline form of claim 29 wherein the crystalline from is stable for one day, one week, two weeks, one month, two months, three months, four months, five months, six months or at least one year during storage at 25° C., 40° C., or 70° C.

31. The crystalline form of claim 29 or 30 wherein less than 10% of the form degrades over a 36 hour period.

32. The crystalline form of any one of claim 1 to 31 wherein the solubility of the crystalline form is at least about 0.25 mg/mL to at least about 10 mg/mL in water or saline.

33. A method of producing the crystalline form of any one of claims 2 to 32, comprising the steps of:
a) reacting psilocin with the acid in a solvent, and
b) drying the resultant product of step a).

34. A pharmaceutical composition comprising the crystalline form of any one of claims 1 to 32.

35. The pharmaceutical composition of claim 34, formulated for oral, subcutaneous, intravenous, or intramuscular administration.

36. The pharmaceutical composition of claim 35, formulated for intravenous administration.

37. A method of treating or preventing a disease or condition in a subject comprising administering to the subject the crystalline form of any one of claims 1 to 32 or the pharmaceutical composition of any one of claims 34 to 36.

38. Use of the crystalline form of any one of claims 1 to 32 or the pharmaceutical composition of any one of claims 34 to 36 in the manufacture of a medicament for treating or preventing a disease or condition.

39. The crystalline form of any one of claims 1 to 32 or the pharmaceutical composition of any one of claims 34 to 36 for use in treating or preventing a disease or condition in a subject.
